SEQUENCE LISTING (107 sequences)

SEQ ID NO: 1 - DNA, 476 bp, Artificial Sequence
  Description of Artificial Sequence Synthetic probe
aatcgctaggccgatttcttaaaccccaaactgttctttgcgagcctgacgcccaaaaccaggggtgtgtagcggccacgtcctttcttaaggctctgggttcccttcccgcttcccgccctccgaccctccaaagcagctttccgccttgctctccggctcccggattccccaggtggccgggggcgcgggtccaacggctctgggaaggcgacttcccggcacctccgggcggcgcgagagcacccttggccctgaactgggccggttgtgtccatccctcgaccccttccctagttaggtgtccttttctgtttttcgaacgaccgggtgatgggtgagcggaaagccgcttccaggagaccaaaagaaaggggtgcctttagaggacgggtgttccccaagggctcggactcaggagtcccagatctccctctttaacttcaccccggttgcgcaattcaaagtctgagggg

SEQ ID NO: 2 - DNA, 20 bp, Artificial Sequence
  Description of Artificial Sequence Synthetic oligonucleotide
agcctgagcacccgtccatg

SEQ ID NO: 3 - DNA, 20 bp, Artificial Sequence
  Description of Artificial Sequence Synthetic oligonucleotide
acacctgcacatggagatcg

SEQ ID NO: 4 - DNA, 20 bp, Artificial Sequence
  Description of Artificial Sequence Synthetic oligonucleotide
ttcaatagatgacctcccca

SEQ ID NO: 5 - DNA, 20 bp, Artificial Sequence
  Description of Artificial Sequence Synthetic oligonucleotide
tgagcgagactgagcaacac

SEQ ID NO: 6 - DNA, 20 bp, Artificial Sequence
  Description of Artificial Sequence Synthetic oligonucleotide
tagacatgattcttcaacag

SEQ ID NO: 7 - DNA, 20 bp, Artificial Sequence
  Description of Artificial Sequence Synthetic oligonucleotide
agttcaggatgaccacataa

SEQ ID NO: 8 - DNA, 20 bp, Artificial Sequence
  Description of Artificial Sequence Synthetic oligonucleotide
tacaacctgtgaccatcagc

SEQ ID NO: 9 - DNA, 20 bp, Artificial Sequence
  Description of Artificial Sequence Synthetic oligonucleotide
gtttcacagcaatgctgacc

SEQ ID NO: 10 - DNA, 20 bp, Artificial Sequence
  Description of Artificial Sequence Synthetic oligonucleotide
catcttgcagaactcgtccc

SEQ ID NO: 11 - DNA, 20 bp, Artificial Sequence
  Description of Artificial Sequence Synthetic oligonucleotide
gcaagatgataagaagcttc

SEQ ID NO: 12 - DNA, 20 bp, Artificial Sequence
  Description of Artificial Sequence Synthetic oligonucleotide
caacctccagatccatcacg

SEQ ID NO: 13 - DNA, 20 bp, Artificial Sequence
  Description of Artificial Sequence Synthetic oligonucleotide
gaccgtcctgccccacgtga

SEQ ID NO: 14 - DNA, 20 bp, Artificial Sequence
  Description of Artificial Sequence Synthetic oligonucleotide
gattgaccttgaaaatacat

SEQ ID NO: 15 - DNA, 20 bp, Artificial Sequence
  Description of Artificial Sequence Synthetic oligonucleotide
ctttctcataatttacagca

SEQ ID NO: 16 - DNA, 20 bp, Artificial Sequence
  Description of Artificial Sequence Synthetic oligonucleotide
tctcctccacgatcacgccc

SEQ ID NO: 17 - DNA, 20 bp, Artificial Sequence
  Description of Artificial Sequence Synthetic oligonucleotide
cggctgggccctgaacatga

SEQ ID NO: 18 - DNA, 20 bp, Artificial Sequence
  Description of Artificial Sequence Synthetic oligonucleotide
gaaacagctcgttgtaccgc

SEQ ID NO: 19 - DNA, 20 bp, Artificial Sequence
  Description of Artificial Sequence Synthetic oligonucleotide
agaacgcatgatagcgtgtc

SEQ ID NO: 20 - DNA, 20 bp, Artificial Sequence
  Description of Artificial Sequence Synthetic oligonucleotide
ctaagaccagctgaaaacca

SEQ ID NO: 21 - DNA, 20 bp, Artificial Sequence
  Description of Artificial Sequence Synthetic oligonucleotide
tcaggaaaaagaagaagtag

SEQ ID NO: 22 - DNA, 20 bp, Artificial Sequence
  Description of Artificial Sequence Synthetic oligonucleotide
ggacactcacatcggtgttt

SEQ ID NO: 23 - DNA, 20 bp, Artificial Sequence
  Description of Artificial Sequence Synthetic oligonucleotide
ccactacaatgtgttcgtag

SEQ ID NO: 24 - DNA, 20 bp, Artificial Sequence
  Description of Artificial Sequence Synthetic oligonucleotide
tggcagaacggaggtgcgcg

SEQ ID NO: 25 - DNA, 20 bp, Artificial Sequence
  Description of Artificial Sequence Synthetic oligonucleotide
tggccggccgtgcagcacgt

SEQ ID NO: 26 - DNA, 20 bp, Artificial Sequence
  Description of Artificial Sequence Synthetic oligonucleotide
caagctggcgtcgcccgagc

SEQ ID NO: 27 - DNA, 20 bp, Artificial Sequence
  Description of Artificial Sequence Synthetic oligonucleotide
gattatcaggcgctccagct

SEQ ID NO: 28 - DNA, 20 bp, Artificial Sequence
  Description of Artificial Sequence Synthetic oligonucleotide
tataaggtgcttctaattgt

SEQ ID NO: 29 - DNA, 20 bp, Artificial Sequence
  Description of Artificial Sequence Synthetic oligonucleotide
tgtggtccactgataatatc

SEQ ID NO: 30 - DNA, 20 bp, Artificial Sequence
  Description of Artificial Sequence Synthetic oligonucleotide
atggtaccaactgctcaaga

SEQ ID NO: 31 - DNA, 20 bp, Artificial Sequence
  Description of Artificial Sequence Synthetic oligonucleotide
tgtgagaactgttgtcagta

SEQ ID NO: 32 - DNA, 20 bp, Artificial Sequence
  Description of Artificial Sequence Synthetic oligonucleotide
gcctacagaccaaatatcaa

SEQ ID NO: 33 - DNA, 20 bp, Artificial Sequence
  Description of Artificial Sequence Synthetic oligonucleotide
gcagtaggtctggtgctcaa

SEQ ID NO: 34 - DNA, 20 bp, Artificial Sequence
  Description of Artificial Sequence Synthetic oligonucleotide
ctcataaagttacatagtca

SEQ ID NO: 35 - DNA, 20 bp, Artificial Sequence
  Description of Artificial Sequence Synthetic oligonucleotide
agaatcagactcatgccaag

SEQ ID NO: 36 - DNA, 20 bp, Artificial Sequence
  Description of Artificial Sequence Synthetic oligonucleotide
acaggtcatcttaatgagcc

SEQ ID NO: 37 - DNA, 20 bp, Artificial Sequence
  Description of Artificial Sequence Synthetic oligonucleotide
ctcagagattgctgcatagt

SEQ ID NO: 38 - DNA, 20 bp, Artificial Sequence
  Description of Artificial Sequence Synthetic oligonucleotide
aaggttctagttgaaacacc

SEQ ID NO: 39 - DNA, 20 bp, Artificial Sequence
  Description of Artificial Sequence Synthetic oligonucleotide
aaccttcagacccaatgtta

SEQ ID NO: 40 - DNA, 20 bp, Artificial Sequence
  Description of Artificial Sequence Synthetic oligonucleotide
agctcgtcttccgccggacc

SEQ ID NO: 41 - DNA, 20 bp, Artificial Sequence
  Description of Artificial Sequence Synthetic oligonucleotide
gcgccgcgtttccagcgtac

SEQ ID NO: 42 - DNA, 20 bp, Artificial Sequence
  Description of Artificial Sequence Synthetic oligonucleotide
aggtggtacctggcccccga

SEQ ID NO: 43 - DNA, 20 bp, Artificial Sequence
  Description of Artificial Sequence Synthetic oligonucleotide
cctgttcacaccccgcagca

SEQ ID NO: 44 - DNA, 20 bp, Artificial Sequence
  Description of Artificial Sequence Synthetic oligonucleotide
ttcccctgcgcgactgccag

SEQ ID NO: 45 - DNA, 20 bp, Artificial Sequence
  Description of Artificial Sequence Synthetic oligonucleotide
ctcaccattggtgtgctgca

SEQ ID NO: 46 - DNA, 20 bp, Artificial Sequence
  Description of Artificial Sequence Synthetic oligonucleotide
tgagcctaccttggcctgga

SEQ ID NO: 47 - DNA, 20 bp, Artificial Sequence
  Description of Artificial Sequence Synthetic oligonucleotide
attccacgggaaggagatct

SEQ ID NO: 48 - DNA, 20 bp, Artificial Sequence
  Description of Artificial Sequence Synthetic oligonucleotide
acggaggctaagcgtcgcaa

SEQ ID NO: 49 - DNA, 20 bp, Artificial Sequence
  Description of Artificial Sequence Synthetic oligonucleotide
cgcttccgcggcccgttcaa

SEQ ID NO: 50 - DNA, 20 bp, Artificial Sequence
  Description of Artificial Sequence Synthetic oligonucleotide
cgatatccatgccctccacc

SEQ ID NO: 51 - DNA, 20 bp, Artificial Sequence
  Description of Artificial Sequence Synthetic oligonucleotide
cttaaacccagttccttcaa

SEQ ID NO: 52 - DNA, 20 bp, Artificial Sequence
  Description of Artificial Sequence Synthetic oligonucleotide
accctggttcactttggacc

SEQ ID NO: 53 - DNA, 20 bp, Artificial Sequence
  Description of Artificial Sequence Synthetic oligonucleotide
ttcatccttaccctcaacga

SEQ ID NO: 54 - DNA, 20 bp, Artificial Sequence
  Description of Artificial Sequence Synthetic oligonucleotide
ctagccggcctagctgtgca

SEQ ID NO: 55 - DNA, 20 bp, Artificial Sequence
  Description of Artificial Sequence Synthetic oligonucleotide
gaaatagcacatactctctg

SEQ ID NO: 56 - DNA, 20 bp, Artificial Sequence
  Description of Artificial Sequence Synthetic oligonucleotide
cacgcgccacaccgtcgatc

SEQ ID NO: 57 - DNA, 20 bp, Artificial Sequence
  Description of Artificial Sequence Synthetic oligonucleotide
atgttatatattgccgatta

SEQ ID NO: 58 - DNA, 20 bp, Artificial Sequence
  Description of Artificial Sequence Synthetic oligonucleotide
ctccaggtatcccatcgaaa

SEQ ID NO: 59 - DNA, 20 bp, Artificial Sequence
  Description of Artificial Sequence Synthetic oligonucleotide
caggtgccttagtgaccacg

SEQ ID NO: 60 - DNA, 20 bp, Artificial Sequence
  Description of Artificial Sequence Synthetic oligonucleotide
ggattaacactgcagagtaa

SEQ ID NO: 61 - DNA, 20 bp, Artificial Sequence
  Description of Artificial Sequence Synthetic oligonucleotide
tgctcctaccaataaaacac

SEQ ID NO: 62 - DNA, 20 bp, Artificial Sequence
  Description of Artificial Sequence Synthetic oligonucleotide
tgggaagcatgtcaccaacc

SEQ ID NO: 63 - DNA, 20 bp, Artificial Sequence
  Description of Artificial Sequence Synthetic oligonucleotide
tctgcttcggatttactaga

SEQ ID NO: 64 - DNA, 20 bp, Artificial Sequence
  Description of Artificial Sequence Synthetic oligonucleotide
atttacgtgaacacacagca

SEQ ID NO: 65 - DNA, 20 bp, Artificial Sequence
  Description of Artificial Sequence Synthetic oligonucleotide
ccacgaacgttcttctctag

SEQ ID NO: 66 - DNA, 20 bp, Artificial Sequence
  Description of Artificial Sequence Synthetic oligonucleotide
ccatcgagtgccaaatgaag

SEQ ID NO: 67 - DNA, 41 bp, Artificial Sequence
  Description of Artificial Sequence Synthetic primer
aatggactatcatatgcttaccgtaacttgaaagtatttcg

SEQ ID NO: 68 - DNA, 44 bp, Artificial Sequence
  Description of Artificial Sequence Synthetic primer
ctttagtttgtatgtctgttgctattatgtctactattctttcc

SEQ ID NO: 69 - DNA, 91 bp, Artificial Sequence
  Description of Artificial Sequence Synthetic primer
  modified_base (59)..(67): a, c, t, g, unknown or other
  misc_feature (59)..(67): This region may encompass 1-9 nucleotides
aatgatacggcgaccaccgagatctacactctttccctacacgacgctcttccgatctnnnnnnnnntcttgtggaaaggacgaaacaccg

SEQ ID NO: 70 - DNA, 89 bp, Artificial Sequence
  Description of Artificial Sequence Synthetic primer
  modified_base (25)..(30): a, c, t, g, unknown or other
caagcagaagacggcatacgagatnnnnnngtgactggagttcagacgtgtgctcttccgatcttctactattctttcccctgcactgt

SEQ ID NO: 71 - DNA, 20 bp, Artificial Sequence
  Description of Artificial Sequence Synthetic primer
agcctcctccatctctttcc

SEQ ID NO: 72 - DNA, 20 bp, Artificial Sequence
  Description of Artificial Sequence Synthetic primer
acttttcaaagccgggtagc

SEQ ID NO: 73 - DNA, 20 bp, Artificial Sequence
  Description of Artificial Sequence Synthetic primer
atgaggatgcgcttgttctc

SEQ ID NO: 74 - DNA, 20 bp, Artificial Sequence
  Description of Artificial Sequence Synthetic primer
cactgtctgaggctcctcct

SEQ ID NO: 75 - DNA, 20 bp, Artificial Sequence
  Description of Artificial Sequence Synthetic primer
gatgagacaagggaccctga

SEQ ID NO: 76 - DNA, 20 bp, Artificial Sequence
  Description of Artificial Sequence Synthetic primer
tgactgcaaagatggaaacg

SEQ ID NO: 77 - DNA, 20 bp, Artificial Sequence
  Description of Artificial Sequence Synthetic primer
agtgagaggcaacctggaga

SEQ ID NO: 78 - DNA, 21 bp, Artificial Sequence
  Description of Artificial Sequence Synthetic primer
catgagtgtggatccagcttg

SEQ ID NO: 79 - DNA, 19 bp, Artificial Sequence
  Description of Artificial Sequence Synthetic primer
tggacagttacgcgcacat

SEQ ID NO: 80 - DNA, 20 bp, Artificial Sequence
  Description of Artificial Sequence Synthetic primer
cgcacggaatttgaacagta

SEQ ID NO: 81 - DNA, 23 bp, Artificial Sequence
  Description of Artificial Sequence Synthetic primer
caacataaacggactcaatccca

SEQ ID NO: 82 - DNA, 20 bp, Artificial Sequence
  Description of Artificial Sequence Synthetic primer
cccacaacacaacctacagc

SEQ ID NO: 83 - DNA, 20 bp, Artificial Sequence
  Description of Artificial Sequence Synthetic primer
tccctcttccctcctcaaat

SEQ ID NO: 84 - DNA, 18 bp, Artificial Sequence
  Description of Artificial Sequence Synthetic primer
gggagcggtgaagatgga

SEQ ID NO: 85 - DNA, 23 bp, Artificial Sequence
  Description of Artificial Sequence Synthetic primer
ggagccaaacgggtcatcatctc

SEQ ID NO: 86 - DNA, 20 bp, Artificial Sequence
  Description of Artificial Sequence Synthetic primer
acactcggaccacatccttc

SEQ ID NO: 87 - DNA, 21 bp, Artificial Sequence
  Description of Artificial Sequence Synthetic primer
cctgaataagcagatccatgg

SEQ ID NO: 88 - DNA, 21 bp, Artificial Sequence
  Description of Artificial Sequence Synthetic primer
cgagtaggacatgctgtaggt

SEQ ID NO: 89 - DNA, 20 bp, Artificial Sequence
  Description of Artificial Sequence Synthetic primer
ggatcagggacctgtcacac

SEQ ID NO: 90 - DNA, 22 bp, Artificial Sequence
  Description of Artificial Sequence Synthetic primer
accacctctacgaacacattgt

SEQ ID NO: 91 - DNA, 20 bp, Artificial Sequence
  Description of Artificial Sequence Synthetic primer
gcgagactgacgcctatgta

SEQ ID NO: 92 - DNA, 20 bp, Artificial Sequence
  Description of Artificial Sequence Synthetic primer
tcagcgtgtaaaggcatctg

SEQ ID NO: 93 - DNA, 22 bp, Artificial Sequence
  Description of Artificial Sequence Synthetic primer
tcatgttgctcacggaggagta

SEQ ID NO: 94 - DNA, 22 bp, Artificial Sequence
  Description of Artificial Sequence Synthetic primer
gaggggccatccacagtcttct

SEQ ID NO: 95 - DNA, 20 bp, Artificial Sequence
  Description of Artificial Sequence Synthetic primer
ctcctggctccaggtgatag

SEQ ID NO: 96 - DNA, 20 bp, Artificial Sequence
  Description of Artificial Sequence Synthetic primer
tccaacagtcccctgatttc

SEQ ID NO: 97 - DNA, 20 bp, Artificial Sequence
  Description of Artificial Sequence Synthetic primer
tggccaggcatggtgtaatc

SEQ ID NO: 98 - DNA, 20 bp, Artificial Sequence
  Description of Artificial Sequence Synthetic primer
tcccctgtgtttggagaaag

SEQ ID NO: 99 - DNA, 20 bp, Artificial Sequence
  Description of Artificial Sequence Synthetic primer
cctcaagctgctcccagata

SEQ ID NO: 100 - DNA, 20 bp, Artificial Sequence
  Description of Artificial Sequence Synthetic primer
atgttggccaggctggtctc

SEQ ID NO: 101 - DNA, 32 bp, Artificial Sequence
  Description of Artificial Sequence Synthetic oligonucleotide
actgcaactatcctgacgtgtgacaggtccct

SEQ ID NO: 102 - DNA, 66 bp, Homo sapiens
ctgcaggagatcatgaagcagacgggctacctgaccatcgggggccaggtaccaccttcactgtgg

SEQ ID NO: 103 - DNA, 56 bp, Artificial Sequence
  Description of Artificial Sequence Synthetic oligonucleotide
ctgcaggagatcatgaagcagacgggctacctggccaggtaccaccttcactgtgg

SEQ ID NO: 104 - DNA, 40 bp, Artificial Sequence
  Description of Artificial Sequence Synthetic oligonucleotide
ctgcaggagatcatgaagccaggtaccaccttcactgtgg

SEQ ID NO: 105 - DNA, 66 bp, Homo sapiens
cccgacgtggggctgctcaagctggcgtcgcccgagctggagcgcctgataatccagtccagcaac

SEQ ID NO: 106 - DNA, 52 bp, Artificial Sequence
  Description of Artificial Sequence Synthetic oligonucleotide
cccgacgtggggctgctcaagctggcgtcgcctgataatccagtccagcaac

SEQ ID NO: 107 - DNA, 49 bp, Artificial Sequence
  Description of Artificial Sequence Synthetic oligonucleotide
cccgacgtggggctgctcaagctggcgcctgataatccagtccagcaac

(19) United States
(12) Patent Application Publication
(10) Pub. No.: US 2019/0224251 A1
(43) Pub. Date: Jul. 25, 2019

(54) METHODS OF DIFFERENTIATING STEM CELLS INTO ENDODERM

(71) Applicant: MEMORIAL SLOAN-KETTERING CANCER CENTER, New York, NY (US)

(72) Inventors: Danwei Huangfu, New York, NY (US); Qing Li, New York, NY (US)

(73) Assignee: MEMORIAL SLOAN-KETTERING CANCER CENTER, New York, NY (US)

(21) Appl. No.: 16/278,933

(22) Filed: Feb. 19, 2019

Related U.S. Application Data
(63) Continuation of application No. PCT/US2017/047599, filed on Aug. 18, 2017.
(60) Provisional application No. 62/377,363, filed on Aug. 19, 2016.

(51) Int. Cl. A61K 35/39; C12N 5/0735; A61P 3/10; A61K 35/545

(52) U.S. Cl. CPC A61K 35/39; C12N 5/0606; A61P 3/10; C12N 2501/16; C12N 2501/727; C12N 2501/999; C12N 2501/415; A61K 35/545

(57) ABSTRACT

The present invention relates to methods and compositions for enhancing the differentiation of stem cells into endodermal cells by inhibiting JNK signaling. The present invention is also directed to methods of treating endodermal disorders in a subject, comprising administering inhibitors of JNK signaling to the subject.

### CROSS-REFERENCE TO RELATED APPLICATION

This application is a Continuation of International Patent Application No. PCT/US2017/047599, filed Aug. 18, 2017, which claims priority to U.S. Provisional Application No. 62/377,363 filed on Aug. 19, 2016, priority to each of which is claimed, and the contents of each of which are incorporated by reference in their entireties herein.

### GRANT INFORMATION

This invention was made with government support under DK096239 and CA008748 awarded by the National Institutes of Health. The government has certain rights in the invention.

### SEQUENCE LISTING

The specification further incorporates by reference the Sequence Listing submitted herewith via EFS on Feb. 19, 2019. Pursuant to 37 C.F.R. § 1.52(e)(5), the Sequence Listing text file, identified as 072734_0836_SL.txt, is 26,081 bytes and was created on Feb. 19, 2019. The Sequence Listing electronically filed herewith, does not extend beyond the scope of the specification and thus does not contain new matter.

### 1. INTRODUCTION

The present invention relates to methods and compositions for differentiating a stem cell into an endoderm cell by inhibiting JNK signaling.

### 2. BACKGROUND OF THE SUBJECT MATTER

Human pluripotent stem cells (hPSCs), such as embryonic stem cells (ESCs) and induced pluripotent stem cells (iPSCs) offer a unique model for studying human gastrulation, as, in vitro, human embryo culturing cannot proceed beyond this stage. Somatic lineage specification occurs at the gastrulation stage of embryogenesis, as epiblast cells reorganize to a trilaminar structure containing ectoderm, mesoderm and definitive endoderm (DE). Components of the Nodal/TGFβ signaling pathway and downstream transcription factors of the GATA and FOXA families are key regulators that initiate DE specification (Tam, 2007; Tsankov, 2015; Zorn, 2009). However, genes that limit generation of DE are largely unknown. Therefore, there remains a need to discover key regulators in limiting generation of DE.

### 3. SUMMARY OF THE INVENTION

The presently disclosed subject matter relates to endoderm cells, and precursors thereof, derived from stem cells (e.g., human stem cells) at least in part by in vitro differentiation.

The presently disclosed subject matter relates to the discovery that endoderm cells, and precursors thereof, can be differentiated from stem cells (e.g., human stem cells) by inhibiting JUN N-terminal Kinase (JNK) pathway signaling. In certain non-limiting embodiments, inhibiting JNK signaling in a stem cell increases the efficiency of differentiation of the stem cell into an endoderm cell in response to endoderm differentiation factors, such as one or more activators of Wingless (Wnt) signaling in combination with one or more activators of Nodal signaling.

In certain non-limiting embodiments, a stem cell (e.g., an embryonic stem cell, a pluripotent embryonic stem cell, or an induced pluripotent stem cell) is contacted with one or more agent(s) that inhibits or reduces JNK signaling, for example, JNK-IN-8, wherein the cells are contacted with the inhibitor in an amount effective to reduce phosphorylation of JUN. In certain embodiments, the level of JNK signaling is reduced by at least about 10, 20 30, 40, 50, 60, 70, 80, 90, 95, 99% or more compared to cells not contacted with the agents.

In certain non-limiting embodiments, the agent that inhibits JNK signaling comprises a nucleic acid that specifically binds to a nucleic acid encoding a protein of the JNK signaling pathway, for example, one or more of MAPK kinase kinase such as mitogen-activated protein kinase kinase kinase 1 (MEKK1), mitogen-activated protein kinase kinase 4 (MKK4) and/or mitogen-activated protein kinase kinase 7 (MKK7); c-Jun N-terminal kinase 1 (JNK1); and/or its substrate transcription factor Jun proto-oncogene (JUN or C-JUN), wherein the binding of the nucleic acid results in a reduction of JNK pathway signaling, for example, by reducing phosphorylation of JUN. In certain non-limiting embodiments, the agent comprises micro RNA (miRNA), interfering RNA (RNAi) molecule, shRNA molecule, antisense RNA, catalytic RNA, and/or catalytic DNA.

In certain non-limiting embodiments, the agent that inhibits JNK signaling comprises an antibody, or antigen binding fragment thereof, that specifically binds to a protein of the JNK signaling pathway, for example, MEKK1, MKK4, MKK7, JNK1, and/or C-JUN.

In certain embodiments, the stem cell or cells are contacted with the foregoing one or more agent(s) in amount(s) effective to increase detectable levels of expression of at least one, two, three, four, five, or six or more markers of endoderm cells, or precursors thereof, for example, but not limited to, SRY-box 17 (SOX17), forkhead box protein A2 (FOXA2), C-X-C motif chemokine receptor 4 (CXCR4), eomesodermin (EMOES), GATA binding protein 4 (GATA4), and/or GATA binding protein 6 (GATA6). In certain embodiments, the level of expression is increased by at least about 5, 10, 20 30, 40, 50, 60, 70, 80, 90, 95, 99% or more compared to cells not contacted with the agents.

In certain embodiments, the cell or cells are further contacted with one or more agents that promote the differentiation of endoderm cells into tissue specific endoderm-derived cell types, for example, pancreatic beta-cells, cells of the gastrointestinal tract, respiratory tract cells, alveolar epithelial cells, lung epithelial cells, endocrine gland cells, and/or cells of the urinary system. In certain embodiments, the cells are further differentiated into organs or tissue thereof, for example, thyroid, esophagus, lung, liver, biliary tree, stomach, pancreas, small intestine, and/or colon.

In certain embodiments, the cell or cells are contacted with the foregoing one or more agent(s) in amount(s) effective to increase detectable levels of expression of at least one, two, or more markers of pancreatic progenitors, for example, but not limited to, NKX6.1, and/or PDX1. In certain embodiments, the level of expression is increased by at least about 5, 10, 20 30, 40, 50, 60, 70, 80, 90, 95, 99% or more compared to cells not contacted with the agents.

In certain embodiments, the cell or cells are contacted with the foregoing agents in amounts effective to increase detectable levels of expression of one or more markers of lung progenitors, for example, but not limited to, NKX2.1. In certain embodiments, the level of expression is increased by at least about 5, 10, 20 30, 40, 50, 60, 70, 80, 90, 95, 99% or more compared to cells not contacted with the agents.

In certain non-limiting embodiments, the cells are differentiated into insulin-secreting β cells. Such cells can be used, for example, in a method of treating type I diabetes.

In certain non-limiting embodiments, the cells are differentiated into alveolar epithelial cells. Such cells can be used, for example, in a method of treating chronic obstructive pulmonary disease (COPD).

The present disclosure also provides for a population of in vitro differentiated cells expressing one or more markers of endoderm cells, or precursors thereof, prepared according to the methods described herein. In certain embodiments, the differentiated cell population is derived from a population of human stem cells. The presently disclosed subject matter further provides for compositions comprising such a differentiated cell population.

In certain embodiments, the population of cells expresses detectable levels of one or more pluripotency marker, for example, OCT4, NANOG, and/or SOX2, as well as one or more endoderm marker. In certain embodiments the marker of pluripotency is expressed by up to about 0.1, 0.5, 1, 5, 10, 20, 30, 40, or 50% of the population of cells.

The presently disclosed subject matter further provides for methods of treating a subject diagnosed with, or at risk for having, a disease or disorder that disrupts the function of endoderm-derived cells, tissues and/or organs, for example diabetes. In certain embodiments, the method comprises administering an effective amount of the differentiated cell population described herein into a subject suffering from said disease or disorder.

In certain embodiments, the present disclosure provides for kits comprising the stem cell-derived precursors prepared according to the methods described herein. In certain non-limiting embodiments, the stem cell-derived cells are endoderm cells. In certain non-limiting embodiments, the cells are mature, differentiated endoderm-derived cells, for example, insulin-secreting β cells. In certain embodiments, the kit can further include instructions, such as a product insert or label, directing the user to utilize the cells for treating a subject diagnosed with, or at risk for having, a disease or disorder that disrupts the function of endoderm-derived cells, tissues and/or organs, for example diabetes.

In certain embodiments, the present disclosure provides for kits comprising one or more agent that can inhibit JNK pathway signaling. In certain embodiments, the kit further comprises one or more endoderm differentiation factors, for example, one or more activators of Wnt signaling and/or one or more activators of Nodal signaling. In certain embodiments, the kit further comprises ESCs or iPSCs. In certain embodiments, the kit can further include instructions, such as a product insert or label, directing the user to utilize the one or more agents to differentiate the ESCs or iPSCs into endoderm cells, or endoderm-derived cells or tissues.

In certain embodiments, the present application also provides for methods of identifying positive and/or negative regulators of endoderm differentiation comprising targeted disruption, for example, inhibition or knock out, of genes in pluripotent cells, for example, ESCs or iPSCs, for example, using CRISPR/Cas gene editing. In certain embodiments, the cells are then differentiated into endoderm cells and expression levels of endoderm markers are detected, wherein detection of expression of an endoderm marker equal to or greater than the level of expression compared to a control (e.g., stem cell-derived endoderm cell that has not been subjected to gene disruption) is indicative of disruption of a negative regulator of endoderm differentiation, and non-detection or detection of a lower level of expression of an endoderm marker compared to a control (e.g., stem cell-derived endoderm cell that has not been subjected to gene disruption) is indicative of disruption of a positive regulator of endoderm differentiation.

The foregoing has outlined the features and technical advantages of the present application in order that the detailed description that follows may be better understood. It should be appreciated by those skilled in the art that the conception and any specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 4. BRIEF DESCRIPTION OF THE FIGURES

FIGS. 1A-1F show a genome-wide screen of positive and negative regulators of DE differentiation. (1A) DE differentiation protocol. CH (CHIR99021, 5 pm), AA (Activin A, 100 ng/ml). (1B) Flow cytometry analysis of SOX17-GFP and CXCR4. Treatment and duration for CH (CHIR99021, 5 pm) and AA (Activin A, 100 ng/ml) are indicated at the top of each flow plot. (1C) Genome-wide CRISPR screen schematic. Each line segment indicates 1 day of media and chemical treatment. (1D) A scatter plot of the gRNA distribution. Y-axis, Z-score of log 2 fold change of SOX17− vs SOX17+. X-axis, the mean abundance of gRNA reads in the SOX17− and SOX17+ populations. Each grey dot represent individual targeting gRNAs. Each black dot represent a non-targeting control gRNAs (1,000 total, built in the library). gRNAs targeting known positive regulators are represents by different shapes and colors, and indicated in the key. Selected positive and negative regulator hits are labeled in green and red, respectively. (1E) Distribution of gRNAs according to Z-scrore. X axis is the Z-score of each gRNA. Y axis is the number of gRNA within the Z-score bin center. gRNA with low reads are left out from the counting, and excluded from further analysis. (1F) Z-score of top 10 positive and top 10 negative regulators. The number next to the gene name indicates the number of gRNA hits. Error bars indicate standard deviation.

FIGS. 2A-2F show validation of top regulators hits from the genome-wide screen. (2A) Schematic showing validation of top hit genes using individual lentivirus expressing different gRNAs. (2B) Bar graphs show the percentage of SOX17+ cells obtained from definitive endoderm differentiation following gRNA targeting. Error bars indicate standard deviation. P value is determined by unpaired two-tailed Student's t test comparing each targeting gRNA to the non-targeting control, where*p<0.05, **p<0.01, ***p<0.001, ****p<0.0001 (n=2). (2C) Histogram overlays show SOX17 expression from flow cytometry analysis. The black lines represent results from control cells infected non-targeting gRNA lentiviruses. The red lines represent results from cells infected with targeting gRNA lentiviruses. Each plot shows results from two non-targeting gRNAs and two different targeting gRNAs, and the experiments were repeated twice. (2D) A table shows a summary of the number of tested and verified regulators. (2E) A schematic view of known positive regulators in TGFβ pathway. (2F) Ranking of top negative regulators based on Z-score.

FIGS. 3A-3M shows characterization of MKK7 and JUN mutant hPSC phenotypes and genetic inactivation of MKK7 or JUN promotes endoderm differentiation. (3A) JNK pathway illustration (left side), SOX17 expression of lenti-CRISPR KO JNK pathway members (right and lower side), light grey line is mutant gRNA, black line is control. (3B) MKK7 and JUN protein structures depicting their important functional domains. The arrows indicate the locations of the gRNA target sequences. The blue diamond squares indicate the location of phosphorylation sites. (3C) Western blot analysis DE cells generated from high AA (100 ng/ml). GAPDH was used as a loading control. (3D) Representative flow cytometry analysis of DE cells in high (100 ng/ml) or low (20 ng/ml) AA conditions. Each plot is a representation of one genotype from 3 independent experiments. (3E). A bar graph summary of flow cytometry analysis of CXCR4+SOX17+ cells in high (100 ng/ml) or low (20 ng/ml) AA conditions. Error bars indicate standard deviation. P value is determined by unpaired two-tailed Student's t test comparing KO to WT cells, where **p<0.01, ***p<0.001, ****p<0.0001 (n=3). (3F) Immunostaining with DE markers SOX17 and FOXA2 of cells differentiated in high (100 ng/ml) or low (20 ng/ml) AA conditions. Scale bar, 100 μm. (3G) Gene expression analysis by RT-qPCR of DE cells generated from high (100 ng/ml) or low (20 ng/ml) AA conditions. The relative expression level is normalized to the housekeeping gene GAPDH. Error bars indicate standard deviation. P value is determined by unpaired two-tailed Student's t test comparing KO to WT cells in high (100 ng/ml) or low (20 ng/ml) AA conditions, where *p<0.05, **p<0.01, ***p<0.001, ****p<0.0001 (n=3). (3H) Representative flow cytometry plot of DE cells in high AA (100 ng/ml) or low AA (5 ng/ml) conditions. (3I) Quantification of SOX17+DE cells in high AA (100 ng/ml) or low AA (5 ng/ml) conditions. n=3. (3J) Immunostaining of SOX17 and FOXA2 of DE cells generated in low AA (5 ng/ml) condition. (3K) qPCR analysis gene expression of DE cells generated from high AA (100 ng/ml) and low AA (5 ng/ml) conditions. Fold change are normalized to WT high AA conditions. n=3. (3L) Western blot analysis of phosphorylation of C-JUN in no AA, high AA (100 ng/ml) or low AA (5 ng/ml) conditions. (3M) JNK signaling pathway constrain endoderm differentiation signaling.

FIGS. 4A-4H show using JNK inhibitor to improve DE differentiation efficiency, and promotes endoderm differentiation. (4A) DE differentiation protocol with JNK-IN-8 treatment plan, lμm IN-8 or equal concentration of DMSO were used for 2-3 days. JNKi indicates the JNK inhibitor JNK-IN-8 (1 uM). (4B) Western blot analysis of H1-derived DE cells at day 3 confirms the inhibition of JUN phosphorylation by JNK-IN-8 (1 μm) treatment. (4C) Flow cytometry analysis of SOX17/GFP+ cells under different Activin A doses. Error bars indicate standard deviation. P value is determined by unpaired two-tailed Student's t test comparing JNK-IN-8 treated samples to untreated control samples, where ***p<0.001, ****p<0.0001 (n=3). (4D) Summary of flow cytometry analysis of SOX17 expression in DE cells from multiple human ES and iPS cell lines. Error bars indicate standard deviation. P value is determined by unpaired two-tailed Student's t test comparing JNK-IN-8 treated samples to untreated control samples, where *p<0.05 **p<0.01, ***p<0.001, ****p<0.0001 (n=3). (4E) Western blotting analysis for the signaling crosstalk between Nodal and JNK pathways. Activin A was used for Nodal activation, while the TGF-β receptor kinase inhibitor SB431542 (TGF-(3i) was used to inhibit the Nodal pathway. JNK-IN-8 was used to block the JNK pathway. (4F) ChIP-qPCR analysis of SMAD2/3 and JUN binding at the SOX17 and GATA6 enhancers. IgG antibody was used as a readout for non-specific background signals. A negative control region at a non-SMAD binding site near the SOX17 enhancer was used as an internal negative control for ChIP-qPCR. Error bars indicate standard deviation. P value is determined by unpaired two-tailed Student's t test comparing JNK-IN-8 treated samples to untreated control samples, where *p<0.05 (n=3). (4G) A summary of the proposed model. Genes in green are validated positive regulators. Genes in red are validated negative regulators. Definitive endoderm differentiation is driven by the Nodal/Activin A-Smad2 pathway, and constrained by the JNK-JUN pathway. Inhibition of the JNK pathway by a JNK inhibitor blocks the phosphorylation of JUN and allows more efficient binding of the SMAD complex to the SOX17 enhancer. (4H) Summary of flow cytometry analysis of SOX17 in H1 cell line and SOX17GFP in HUES8-SOX17GFP reporter at Day2 and Day3.

FIGS. 5A-5G show establishment of HUES8 SOX17^{GFP/+} iCas9 cells for genome-wide screen. (5A) Schematic shows iCRISPR platform used for efficient genome editing. Doxycycline inducible Cas9 cells were transduced with lentiviruses expressing gRNAs to mediate genome editing. (5B) SOX17^{GFP/+} reporter gene targeting schematic. The knockin strategy for generating the SOX17^{GFP/+} reporter cell line. PAM sequences are labeled in purple, gRNA sequences are labeled in green. FIG. 5B discloses SEQ ID NO: 101. (5C) Southern blot analysis of SOX17^{GFP/+} cell line. 5′ external probe was used to detect the integration of GFP fragment at one of the SOX17 alleles. (5D) Imunnostaining of GFP, SOX17, and FOXA2 of DE cells differentiated from SOX17^{GFP/+} iCAS9 cell lines. Scale bar, 100 μm. (5E) Flow cytometry analysis of co-staining of SOX17 and GFP of DE cells differentiated from SOX17^{GFP/+} iCAS9 cell lines. (5F) Flow cytometry analysis of DE cells differentiated from SOX17^{GFP/+} cells infected with EOMES-gRNA lentivirus (at MOI 0.36). The control sample (Ctrl) is DE cells from uninfected wild-type SOX17^{GFP/+} iCAS9 cell. (5G) Titration of the effect of Activin A and CHIR99021 concentration on DE differentiation efficiency (percentage of SOX17-GFP positive (SOX17^{GFP/+}) cells as a read out)

FIGS. 6A-6F show analysis of genome-wide screen results. (6A) Method of calculating of Z-score of each gRNA from raw read counts. STDEV: standard deviation. (6B, 6F) Counts of Z-score distribution for non-targeting gRNA. (6C) Method of calculating and ranking the Z-score of each hit gene. (6D) Gene ontology analysis of positive regulators gene hits. (6E) Gene ontology analysis of negative regulators gene hits.

FIG. 7 shows top 50 positive and negative regulator gene hits identified from the genome-wide screen. The top 50 positive and negative regulator hits are listed based on the Z-score. Genes in blue were individually tested, and genes marked by asterisks were successfully-verified.

FIG. 8 shows validation of top hit genes from the genome-wide screen. Representative flow plots of DE cells of each lenti-gRNA mutant. Gated in SOX17 and EOMES.

FIGS. 9A-9J show generation and phenotype of MKK7 and JUN KO ESC lines. (9A) gRNA gene targeting strategy of MKK7 KO (i.e., MKK7−/−) and JUN KO (i.e., JUN−/−) lines. Two homozygous knockout cells were picked for further analysis. The blue bars indicate the exons of the gene (JUN is an intronless gene). PAM sequences are labeled in purple. gRNA targeting sequences are labeled in green. FIG. 9A discloses SEQ ID NOS 102-107, respectively, in order of appearance. (9B) Immunostaining of OCT4, NANOG and SOX 2 in WT, MKK7 KO and JUN KO ES cells. Scale bar, 100 pm. (9C) RT-qPCR analysis of DE marker genes in undifferentiated WT, MKK7 KO and JUN KO ES cells. The relative expression level is normalized to the housekeeping gene GAPDH. Error bars indicate standard deviation. P value is determined by unpaired two-tailed Student's t test comparing KO to WT cells. NS (not significant) indicates that the p value is great than 0.05. (9D) Representative flow cytometry analysis (left panel) of DE cells in high AA (100 ng/ml) and low AA (20 ng/ml) conditions. Bar graph (right panel) shows summaries of flow cytometry analysis of GATA6/GATA4+ cells in high AA or low AA condition. Error bars indicate standard deviation. P value is determined by unpaired two-tailed Student's t test comparing KO to WT cells, where **p<0.01, ***p<0.001, ****p<0.0001 (n=3). (9E) RT-qPCR analysis of pluripotency genes and DE marker genes in WT and MKK7−/− hESC. Fold changes are normalized to WT hESC. (9F) RT-qPCR analysis of pluripotency genes in WT and MKK7−/− DE cells. Fold changes are normalized to WT DE cells. High AA, 100 ng/ml. Low AA, 5 ng/ml. (9G) Growth curve of WT, MKK7 KO and JUN KO cells cultured in the self-renewing condition (ESC) or endoderm differentiation condition (DE). For the ESC condition, cell numbers are normalized to Day 0 (1 day after splitting). For the DE condition, cell numbers are normalized to Day 0 (the day when DE differentiation was initiated). Error bars indicate standard deviation. P value is determined by unpaired two-tailed Student's t test comparing KO to WT cells. NS indicates that the p value is great than 0.05 (n=3). (9H) Flow cytometry analysis of proliferation marker Phospho Histone H3 and the apoptosis marker Cleaved Caspase 3 from WT, MKK7 KO and JUNKO cells on Day 3 of DE differentiation. Error bars indicate standard deviation. P value is determined by unpaired two-tailed Student's t test comparing KO to WT cells. NS indicates p value is great than 0.05 (n=3). (9I) Growth curve of WT, MKK7−/− and JUN−/− ESC and DE cells. Cell numbers are normalized to Day 1. (9J) Proliferation marker Phospho Histone H3 and apoptosis marker Cleaved Caspase 3 staining in WT, MKK7−/− JUN−/− (high AA, 100 ng/ml) DE cells, quantification by flow cytometry. N=3.

FIGS. 10A-10D show Neuroectoderm (NE) differentiation of MKK7 and JUN KO ESC lines. (10A) Dual-Smad NE differentiation schematic. The day when NE differentiation was initiated is designated as Day 0. Cells were examined on Day 4, 6, 8 and 10 of NE differentiation. (10B) Immunostaining of PAX6, SOX1 and OCT4 of Day 10 WT, MKK7 KO and JUN KO ESCs. (10C) Representative flow plot of PAX6 of Day 10 WT, MKK7 KO, and JUN KO ESCs. (10D) Quantification of percentage of PAX6 positive cell during NE differentiation from Day 4 to Day 10 based on flow cytometry analyses. Error bars indicate standard deviation. P value is determined by unpaired two-tailed Student's t test comparing KO to WT cells. NS indicates that the p value is great than 0.05. n=3.

FIGS. 11A-11D show the effect of JNK inhibitor JNK-IN-8 on DE differentiation. (11A) Representative flow cytometry plots for CXCR4/S0X17 (top) and GATA6/GATA4 (bottom) in DE cells differentiated from control or JNK-IN-8 treatment in high or low AA conditions as indicated. JNKi indicates the JNK inhibitor JNK-IN-8 (1 uM). (11B) Bar graphs show summaries flow cytometry analysis of CXCR4/S0X17+(top) and GATA6/GATA4+(bottom) cells in high or low AA condition. Error bars indicate standard deviation. P value is determined by unpaired two-tailed Student's t test comparing JNKi treated samples to untreated control samples, where ***p<0.001, ****p<0.0001 (n=3). (11C) Gene expression analysis by RT-qPCR of DE marker genes in DE cells differentiated from control and JNK-IN-8 treated cells in high or low AA conditions (n=3). Error bars indicate standard deviation. P value is determined by unpaired two-tailed Student's t test comparing JNK-IN-8 treated samples to untreated control samples in high or low AA condition, where *p<0.05, **p<0.01, ″*p<0.001, ****p<0.0001 (n=3). (11D) Published ChIP-Seq analysis (Kim, 2011) of SMAD2/3 at the SOX17 and GATA6 locus.

FIG. 12 shows the structure of the JNK inhibitor JNK-IN-8, and the the compound's IC50 for inhiniting JNK1, JNK2, and JNK3.

FIGS. 13A-13E show transient inhibition of JNK pathway improved differentiation efficiency of endoderm derivatives. (13A) Schematic of differentiation to pancreatic and lung lineage with transient inhibition of JNK pathway during definitive endoderm differentiation. (13B) Representative flow cytometry analysis of pancreatic lineage markers PDX1 and NKX6.1. (13C) Quantification of NKX6.1+PDX1+ cells based on flow cytometry analysis. Error bars indicate standard deviation. P value is determined by unpaired two-tailed Student's t test comparing JNKi treated samples to untreated control samples. *p<0.05 (n=3). (13D) Immunostaining with pancreatic lineage markers PDX1 and NKX6.1 of cells differentiated with or without JNKi. Scale Bar, 100 μm. (13E) Representative flow cytometry analysis of lung lineage markers NKX2.1 (left). Quantification of NKX2.1+ cells (right) based on flow cytometry analysis. Error bars indicate standard deviation. P value is determined by unpaired two-tailed Student's t test comparing JNKi treated samples to untreated control samples. *p<0.05 (n=2).

### 5. DETAILED DESCRIPTION OF THE INVENTION

The present invention relates to methods for inducing differentiation of pluripotent stem cells to cells that express one or more endoderm markers, compositions of cells expressing such markers, and methods for treating diseases or disorders of endoderm-derived cells, tissues and/or organs. It is based, at least in part, on the discovery that inhibiting JNK signaling in stem cells (e.g., human stem cells) increases the efficiency in which the cells differentiate into endoderm marker expressing cells when exposed to endoderm differentiation factors such as a Wnt activator (e.g., CHIR99021) and an activator of Nodal signaling (e.g., Activin A). Non-limiting embodiments of the invention are described by the present specification and Examples.

For purposes of clarity of disclosure and not by way of limitation, the detailed description is divided into the following subsections:

5.1. Definitions;

5.2. Method of Differentiating Stem Cells;

5.3. Method of Treatment;

5.4 Compositions Comprising Differentiated Cell Populations; and

5.5. Kits.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “signaling” refers to a “signal transduction protein” refers to a protein that is activated or otherwise affected by ligand binding to a membrane receptor protein or some other stimulus. Examples of signal transduction protein include, but are not limited to, JNK, transforming growth factor beta (TGFβ), Activin, Nodal, and glycogen synthase kinase 3β (GSK3 β) proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., Activin, Nodal, Wnt, etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of a protein or pathway. An inhibitor can be any compound or molecule that changes any activity of a named protein (signaling molecule, any molecule involved with the named signaling molecule, a named associated molecule) (e.g., including, but not limited to, the signaling molecules described herein), for one example, via directly contacting the signaling protein, contacting mRNA, causing conformational changes of the protein, decreasing protein levels, or interfering with interactions with signaling partners (e.g., including those described herein), and affecting the expression of target genes (e.g. those described herein). Inhibitors also include molecules that indirectly regulate biological activity by intercepting upstream signaling molecules (e.g., within the extracellular domain). Antibodies that block upstream or downstream proteins are contemplated for use to neutralize extracellular activators of protein signaling, and the like. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators,” as used herein, refer to compounds that increase, induce, stimulate, activate, facilitate, or enhance activation the signaling function of the molecule or pathway, e.g., Wnt signaling, Nodal signaling, etc.

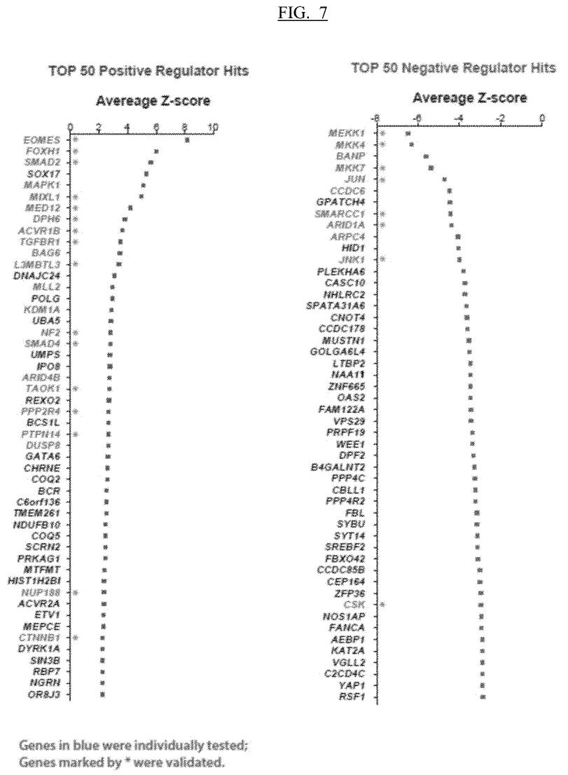

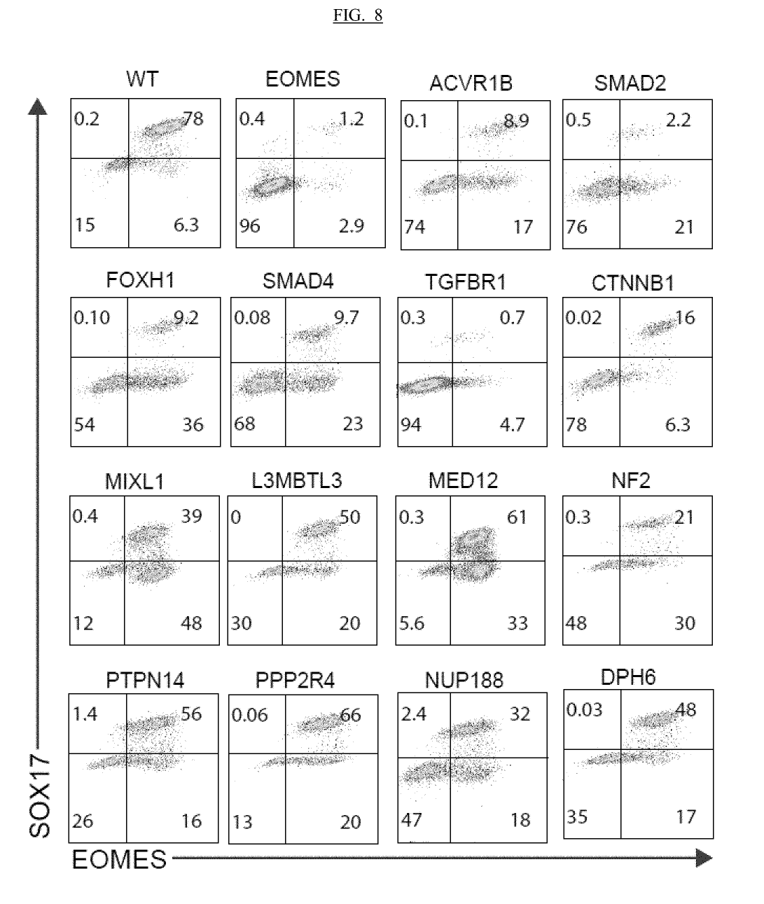

As used herein, the term “positive regulators” refers to proteins that reducing the expression of which can decrease the efficiency of stem cells differentiating into endoderm cells. For instance, FIG. 7 lists positive regulators identified in Example 1.

As used herein, the term “negative regulators” refers to proteins that reducing the expression of which can increase the efficiency of stem cells differentiating into endoderm cells. For instance, FIG. 7 lists negative regulators identified in Example 1. In certain embodiments, MEKK1, MKK4, MKK7, JNK1, and C-JUN are negative regulators.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of endoderm cells, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells. In certain embodiments, the stem cells are human stem cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “pluripotent” refers to an ability to develop into the three developmental germ layers of the organism including endoderm, mesoderm, and ectoderm. In certain embodiments, the pluripotent cell is selected from the group consisting of selected from the group consisting of human, nonhuman primate or rodent non-embryonic stem cells; human, nonhuman primate or rodent embryonic stem cells; human, nonhuman primate or rodent induced pluripotent stem cells; and human, nonhuman primate or rodent recombinant pluripotent cells.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell, for examples, CI 4, C72, and the like. An induced pluripotent stem cell may be prepared from any fully (e.g., mature or adult) or partially differentiated cell using methods known in the art. For example, but not by way of limitation, an induced pluripotent stem cell may be prepared from a fibroblast, such as a human fibroblast; an epithelial cell, such as a human epithelial cell; a blood cell such as a lymphocyte or hematopoietic cell or cell precursor or myeloid cell, such as a human lymphocyte, hematopoietic cell or cell precursor or human myeloid cell; or a renal epithelial cell, such as a human renal epithelial cell. In certain non-limiting embodiments, an induced pluripotent stem cell contains one or more introduced reprogramming factor associated with producing pluripotency. In certain non-limiting embodiments a human induced pluripotent stem cell is not identical to a human embryonic pluripotent stem cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

As used herein, the term “undifferentiated” refers to a cell that has not yet developed into a specialized cell type.

As used herein, the term “differentiation” refers to a process whereby an unspecialized cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as endoderm cells. As used herein, the term “directed differentiation” in reference to a stem cell refers to the use of small molecules, growth factor proteins, and other growth conditions to promote the transition of a stem cell from the pluripotent state into a more mature or specialized cell fate (e.g., endoderm).

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., stem cell) to divide into progeny cells with characteristics that are different from the stem cell, such as genotype (e.g., change in gene expression as determined by genetic analysis such as a microarray) and/or phenotype (e.g., change in expression of a protein, such as one or more endoderm markers, including, but not limited to, SOX17, CXCR4, GATA6, GATA4 and FOXA2).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., one or more inhibitor, activator, and/or inducer) refers to exposing or otherwise providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo), or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, protein or RNA expression, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, the term “an effective amount” or “effective amounts” refers to an amount of one or more agents (e.g., inhibitors of JNK signaling) that is sufficient to achieve the desired effects, for example, directing the in vitro differentiating of stem cells into a population of differentiated cells, for example, cells expressing one or more endoderm markers.

### 5.2 Method of Differentiating Stem Cells

The presently disclosed subject matter is based at least in part on the discovery that the efficiency of differentiating stem cells into endoderm cells in vitro can be increased by inhibiting JNK signaling or certain negative regulators of endoderm differentiation in the cells when contacting the cells with endoderm differentiation factors such as one or more activators of Wnt signaling and one or more activators of Nodal signaling. In certain non-limiting embodiments, the negative regulators are selected from a group consisting of molecules listed in FIG. 7 right panel. In certain embodiments, the negative regulators are selected from the group consisting of MEKK1, MKK7, MKK4, JNK1, C-JUN, SWI/SNF related, matrix associated, actin dependent regulator of chromatin subfamily c member 1 (SMARCC1), AT-rich interaction domain 1A (ARID1A), and C-terminal Src kinase (CSK).

In certain non-limiting embodiments, the agent that inhibits JNK signaling comprises an inhibitor that is selective for one or more of MEKK1, MKK4, MKK7, JNK1, and/or C-JUN. In certain non-limiting embodiments, the inhibitor reduces phosphorylation of JUN.

In certain non-limiting embodiments, the agent comprises JNK-IN-8. In certain embodiments, JNK-IN-8 has CAS number 1410880-22-6, and has the following structure:

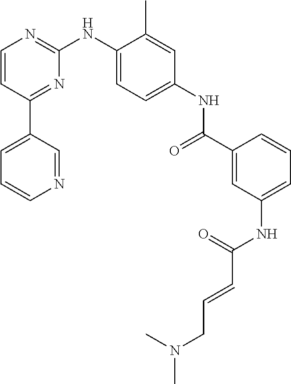 [SMILES: Cc1cc(NC(=O)c2cccc(NC(=O)/C=C/CN(C)C)c2)ccc1Nc1nccc(-c2cccnc2)n1]

In certain non-limiting embodiments, the agent comprises any one or more JNK inhibitors described by Zhang et al., Chem Biol. 2012 Jan. 27; 19(1):140-54, which is incorporated by reference in its entirety.

In certain non-limiting embodiments, the JNK inhibitor is selected from the group consisting of SP600125 (1,9-Pyrazoloanthrone, CAS No. 129-56-6), JNK Inhibitor IX (N-(3-cyano-4,5,6,7-tetrahydrobenzo[b]thien-2-yl)-1-naphthalenecarboxamide, CAS No. 312917-14-9), DTP3 ((R)-2-((R)-2-acetamido-3-(4-hydroxyphenyl)propanamido)-N—((R)-1-amino-1-oxo-3-phenylpropan-2-yl)-5-guanidinopentanamide), and combinations thereof.

In certain non-limiting embodiments, the agent comprises a nucleic acid that specifically binds to a nucleic acid encoding a protein of the JNK signaling pathway, for example, one or more of MEKK1, MKK4, MKK7, JNK1, and/or C-JUN, and reduces JNK signaling and/or phosphorylation of JUN. In certain embodiments, the agent comprises micro RNA (miRNA), interfering RNA (RNAi) molecule, shRNA molecule, antisense RNA, catalytic RNA, and/or catalytic DNA.

In certain non-limiting embodiments, the agent that inhibits JNK signaling comprises an antibody, or antigen binding fragment thereof, that specifically binds to a protein of the JNK signaling pathway, for example, MEKK1, MKK4, MKK7, JNK1, and/or C-JUN.

In certain non-limiting embodiments, the methods of the present invention comprise contacting a stem cell, for example but not limited to, a human ESC or iPSC, with an agent that inhibits JNK signaling, in an amount effective to increase the detectable level of expression of one, two, three, four, five or six or more endodermal markers in the cells. In certain non-limiting embodiments, the endoderm markers include one or more of SOX17, FOXA2, CXCR4, EMOES, GATA4, and/or GATA6.

In certain non-limiting embodiments, the agent is contacted to a plurality of stem cells in an amount effective to increase expression of the one or more endodermal markers in at least, or in up to, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the cells. In certain embodiments, the cells are contacted with the agent for at least, or up to, 1, 2, 3, 4 or 5 days or more, or for at least or up to 24, 48, 72, 96, or 120 hours. In certain embodiments, the cells are contacted with the agent for about 1 day. In certain embodiments, the cells are contacted with the agent for about 2 days. In certain embodiments, the cells are contacted with the agent for about 3 days. In certain embodiments, the cells are contacted with the agent for about 72 hours.

In certain non-limiting embodiments, the agent is contacted to a plurality of stem cells in an amount effective to increase co-expression of SOX17 and CXCR4. In certain non-limiting embodiments, the agent is contacted to a plurality of stem cells in an amount effective to increase co-expression of SOX17 and EOMES. In certain non-limiting embodiments, the agent is contacted to a plurality of stem cells in an amount effective to increase co-expression of SOX17 and FOXA2. In certain non-limiting embodiments, the agent is contacted to a plurality of stem cells in an amount effective to increase co-expression of SOX17, CXCR4, FOXA2 and EOMES.

In certain non-limiting embodiments, the agent is contacted to a plurality of stem cells in an amount effective to increase binding of SMAD2 and/or SMAD3 to their transcriptional targets, for example, enhancer regions of SOX17 and/or GATA6.

In certain non-limiting embodiments, the agent is contacted to a plurality of stem cells in an amount effective to inhibit or reduce binding of JUN to its transcriptional targets, for example, enhancer regions of SOX17 and/or GATA6.

In certain embodiments, the cells are contacted with the agent at a concentration of between about 0.25 and 10 μM, between about 0.5 and 9 μM, between about 1 and 8 μM, between about 1.5 and 7 μM, between about 2 and 6 μM, between about 2.5 and 5 μM, between about 3 and 4 μM, between about 0.5 and 2 μM, or about 1 μM.

In certain embodiments, the cells are contacted with the agent at a concentration of at least about 0.1, 0.5, 1, 1.5, 2, 2.5, 3, 3.5, 4, 4.5, or 5 μM or more.

In certain embodiments, the cells are contacted with the agent at a concentration of between about 0.25 and 50 μM, between about 0.5 and 40 μM, between about 1 and 30 μM, between about 1.5 and 25 μM, between about 2 and 20 μM, between about 2.5 and 15 μM, between about 3 and 10 μM, between about 3.5 and 8 μM, between about 4 and 6 μM, about 4.7 μM, or about 18.7 μM.

In certain embodiments, the cells are also contacted with one or more endoderm differentiation factors, for example, as described by Zhu et al., Cell Stem Cell 18, 755-768 (2016); and/or Tan et al., Stem Cells and Development 22, 1893-1906 (2013), each of which is incorporated by reference in its entirety herein.

In certain embodiments, the cells are contacted with an activator of Wnt signaling, for example but not limited to, Wnt3A, Wnt1, and/or CHIR99021 (aminopyrimidine; or 3-[3-(2-Carboxyethyl)-4-methylpyrrol-2-methylidenyl]-2-indolinone; or 6-(2-(4-(2,4-dichlorophenyl)-5-(4-methyl-1H-imidazol-2-yl)pyrimidin-2-ylamino) ethylamino)nicotinonitrile)), which is an inhibitor of GSK3β.

In certain embodiments, the cells are contacted with an activator of Nodal signaling, for example but not limited to Activin A.

In certain embodiments, the cells are contacted with the Wnt activator for up to, at least, or about 1 or 2 days, or up to, at least, or about 24 or 48 hours; and contacted with the activator of Nodal signaling for up to, at least, or about 1, 2, 3, 4 or 5 days, or up to, at least, or about 24, 48, 72, 96 or 125 hours.

In certain embodiments, the cells are contacted with the activator of Wnt signaling at a concentration of between about 0.5 and 10 μM, between about 1 and 9 μM, between about 2 and 8 μM, between about 3 and 7 μM, between about 4 and 6 μM, or about 5 μM.

In certain embodiments, the cells are contacted with the activator of Wnt signaling at a concentration of at least about 0.1, 0.5, 1, 1.5, 2, 2.5, 3, 3.5, 4, 4.5, 5, 5.5, 6, 6.5, 7, 7.5, 8, 8.5, 9, 9.5, or 10 μM, or more.

In certain embodiments, the cells are contacted with the activator of Nodal signaling at a concentration of between about 0.5 and 200 ng/mL, between about 5 and 100 ng/mL, between about 10 and 90 ng/mL, between about 20 and 80 ng/mL, between about 30 and 70 ng/mL, between about 40 and 80 ng/mL, between about 50 and 70 ng/mL, between about 5 and 60 ng/mL, between about 90 and 110 ng/mL, between about 10 and 30 ng/mL, between about 2 and 10 ng/mL, about 100 ng/mL, about 20 ng/ml, or about 5 ng/mL.

In certain embodiments, the cells are contacted with the activator of Nodal signaling at a concentration of at least about 0.5, 1, 2, 3, 4, 5, 6, 7, 8, 9, 10, 20, 30, 40, 50, 60, 70, 80, 90, or 100 ng/mL or more.

In certain non-limiting embodiments, the stem cell or a progeny cell thereof contains an introduced heterologous nucleic acid, where said nucleic acid may encode a desired nucleic acid or protein product or have informational value (see, for example, U.S. Pat. No. 6,312,911, which is incorporated by reference in its entirety). Non-limiting examples of desired protein products include markers detectable via in vivo imaging studies, for example receptors or other cell membrane proteins such as but not limited to the human sodium iodine symporter.

Non-limiting examples of markers further include fluorescent proteins (such as green fluorescent protein (GFP), blue fluorescent protein (EBFP, EBFP2, Azurite, mKalamal), cyan fluorescent protein (ECFP, Cerulean, CyPet, mTurquoise2), and yellow fluorescent protein derivatives (YFP, Citrine, Venus, YPet, EYFP)), β-galactosidase (LacZ), chloramphenicol acetyltransferase (cat), neomycin phosphotransferase (neo), enzymes (such as oxidases and peroxidases), and antigenic molecules. As used herein, the terms “reporter gene” or “reporter construct” refer to genetic constructs comprising a nucleic acid encoding a protein that is easily detectable or easily assayable, such as a colored protein, fluorescent protein such as GFP or an enzyme such as beta-galactosidase (lacZ gene). In certain embodiments, the reporter can be driven by a recombinant promoter of an endoderm marker gene, for example, SOX17. In certain embodiments, the marker is introduced into the cells using the CRISPR/CAS system and a suitable guide RNA (gRNA).

In certain non-limiting embodiments, the stem cell, or a progeny cell thereof, contains an introduced heterologous nucleic acid that increases or decreases the metabolic processes of the cell, for example, glucose metabolism and/or choline metabolism, wherein the cell can be imaged in vivo using Positron Emission Tomography (PET) due to the altered metabolic activity.

### 5.3 Method of Treatment

The in vitro differentiated cells that express one or more endoderm markers, or precursor thereof, can be used for treating a disease or disorder of endoderm-derived cells, tissues or organs (i.e., endodermal disorders). The presently disclosed subject matter provides for methods of treating an endodermal disorder comprising administering an effective amount of the presently disclosed stem-cell-derived endodermal cells into a subject suffering from an endodermal disorder.

Non-limiting examples of endodermal disorders include diseases and disorders that affect the lung, liver, biliary tree, stomach, intestine, colon, pancreas, gastrointestinal tract, thyroid and/or thymus of a subject. In certain non-limiting embodiments, the subject requires a lung, liver, biliary tree, stomach, intestine, colon, pancreas, gastrointestinal tract, thyroid and/or thymus organ transplant. In certain non-limiting embodiments, the disease or disorder is cystic fibrosis, Chronic obstructive pulmonary disease (COPD), Alpha-1 antitrypsin deficiency, Interstitial Lung Disease (ILD), Bronchiectasis, liver cirrhosis, acute liver failure (ALF), chronic liver failure, end-stage liver disease, biliary atresia, Alagille syndrome, primary biliary cirrhosis, and primary sclerosing cholangitis, hemochromatosis, Wilson disease, nonalcoholic steatohepatitis, Crohn's disease, inflammatory bowel disease, pancreatitis, hyperthyroidism, hypothyroidism, Graves' disease, cancer, or diabetes. In certain non-limiting embodiments, the diabetes is, for example, type I diabetes.

The presently disclosed stem-cell-derived endodermal cells can be administered or provided systemically or directly to a subject for treating or preventing an endodermal disorder. In certain embodiments, the presently disclosed stem-cell-derived endodermal cells are directly injected into an organ of interest (e.g., the liver or pancreas). In certain embodiments, the presently disclosed stem-cell-derived endodermal cells are administered systemically.

The presently disclosed stem-cell-derived endodermal cells can be administered in any physiologically acceptable vehicle. Pharmaceutical compositions comprising the presently disclosed stem-cell-derived endodermal cells and a pharmaceutically acceptable vehicle are also provided. The presently disclosed stem-cell-derived endodermal cells and the pharmaceutical compositions comprising said cells can be administered via localized injection, orthotopic (OT) injection, systemic injection, intravenous injection, or parenteral administration. In certain embodiments, the presently disclosed stem-cell-derived endodermal cells are administered to a subject suffering from diabetes, for example, type I diabetes.

The presently disclosed stem-cell-derived endodermal cells and the pharmaceutical compositions comprising said cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived endodermal cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived endodermal cells. This will present no problem to those skilled in chemical and pharmaceutical principles, or problems can be readily avoided by reference to standard texts or by simple experiments (not involving undue experimentation), from this disclosure and the documents cited herein.

In certain non-limiting embodiments, the cells and precursors described herein are comprised in a composition that further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into mature adult cells, for example, insulin-secreting cells.

One consideration concerning the therapeutic use of the presently disclosed stem-cell-derived endodermal cells is the quantity of cells necessary to achieve an optimal effect. An optimal effect includes, but is not limited to, repopulation of regions of a subject suffering from an endodermal disorder, and/or improved function of the subject's endodermal-derived cells, tissues and organs.

In certain embodiments, an effective amount of the presently disclosed stem-cell-derived endodermal cells is an amount that is sufficient to repopulate regions and organs affected by an endodermal disorder. In certain embodiments, an effective amount of the presently disclosed stem-cell-derived endodermal cells is an amount that is sufficient to improve the function of the endodermal-derived cells, tissues or organs of a subject suffering from an endodermal disorder, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's endodermal-derived cells, tissues or organs.

The quantity of cells to be administered will vary for the subject being treated. The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiments, the cells that are administered to a subject suffering from an endodermal disorder are a population of cells that are differentiated/matured from the presently disclosed stem-cell-derived endodermal cells.

### 5.4 Compositions Comprising Differentiated Cell Populations

The presently disclosed subject matter provides compositions comprising a population of differentiated endoderm cells produced by the in vitro differentiation methods described herein. In certain non-limiting embodiments, the differentiated endoderm cells are prepared from embryonic pluripotent stem cells, such as human embryonic pluripotent stem cells. In certain non-limiting embodiments, the differentiated endoderm cells are prepared from induced pluripotent stem cells, such as induced human pluripotent stem cells.

Furthermore, the presently disclosed subject matter provides compositions comprising a population of in vitro differentiated cells, wherein at least about 70% (e.g., at least about 75%, at least about 80%, at least about 85%, at least about 90%, at least about 95%, at least about 99%, or at least about 99.5%) of the population of cells express one or more endoderm marker, and wherein less than about 15% (e.g., less than about 10%, less than about 5%, less than about 4%, less than about 3%, less than about 2%, less than about 1%, less than about 0.5%, or less than about 0.1%) of the population of cells express one or more marker selected from the group consisting of peripheral sensory neuron markers, nociceptor markers, mechanoreceptor markers, proprioceptor markers, stem cell markers, CNS markers, Cranial Neural Crest (CNC) markers, Melanocyte-competent Neural Crest (MNC) markers, enteric neuron markers, neuronal cell markers, and mesenchymal precursor markers (for example, as described by International Publication Nos. WO/2010/096496, WO/2011/149762, WO/2013/067362, WO/2016/196661, WO/2014/176606, and WO/2015/077648, the contents of each of which are incorporated by reference in their entireties).

Non-limiting examples of endoderm markers include SOX17, FOXA2, CXCR4, EMOES, GATA4, and GATA6.

Non-limiting examples of proprioceptor markers include TrkC, RUNX3, CDHL1, ETV1, and ETV4.

Non-limiting examples of peripheral sensory neuron markers include Brn3A, peripherin, and ISL1.

Non-limiting examples of nociceptor markers include TrkA and RUNX1.

Non-limiting examples of mechanoreceptor markers include TrkB and RET.

Non-limiting examples of stem cell markers include OCT4, NANOG, SOX2, LIN28, SSEA4 and SSEA3.

Non-limiting examples of CNS markers include PAX6, NESTIN, Vimentin, FOXG1, SOX2, TBR1, TBR2 and SOX1.

Non-limiting examples of neuronal cell markers include TUJ1, MAP2, NFH, BRN3A, ISL1, TH, ASCL1, CHAT, PHOX2B, PHOX2A, TRKA, TRKB, TRKC, SHT, GABA, NOS, SST, TH, CHAT, DBH, Substance P, VIP, NPY, GnRH, and CGRP.

Non-limiting examples of mesenchymal precursor markers are SMA, Vimentin, HLA-ABC, CD105, CD90 and CD73.

Non-limiting examples of CNC markers include PAX6, NESTIN, Vimentin, FOXG1, SOX2, TBR1, TBR2 and SOX1.

Non-limiting examples of MNC markers include PAX6, NESTIN, Vimentin, FOXG1, SOX2, TBR1, TBR2 and SOX1.

In certain embodiments, the composition comprises a population of from about 1×10⁴ to about 1×10¹⁰ from about 1×10⁴ to about 1×10⁵ from about 1×10⁵ to about 1×10⁹ from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of cells expressing one or more endoderm marker. In certain embodiments, the composition comprises a population of from about 1×10⁵ to about 1×10⁷ of cells expressing one or more endoderm marker.

In certain non-limiting embodiments, the composition further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain non-limiting embodiments, an endoderm cell produced according to the invention expresses a detectable marker at a level not expressed in a counterpart naturally-derived endoderm cell; said detectable marker may be an endogenous molecule, such as a nucleic acid or protein, or may be exogenous.

In certain embodiments, the composition is a pharmaceutical composition that comprises a pharmaceutically acceptable carrier, excipient, diluent or a combination thereof. In certain embodiments, the compositions can be used for preventing and/or treating disease or disorders as described herein.

### 5.5 Kits

The presently disclosed subject matter provides for kits for inducing differentiation of stem cells. In certain embodiments, the kit comprises one or more of (a) one or more inhibitor of JNK signaling, (b) one or more activator of Wnt signaling, (c) one or more activator of Nodal signaling, (d) instructions for inducing differentiation of the stem cells into a population of differentiated cells that express one or more endodermal marker, or precursor thereof.

In certain embodiments, the instructions comprise contacting the stem cells with the inhibitor(s), activator(s) and molecule(s) in a specific sequence. In certain embodiments, the instructions comprise contacting the stem cells with the inhibitor(s), activator(s) and molecule(s) as described by the methods of the present disclosure (see, supra, Section 5.2).

In certain embodiments, the present disclosure provides for kits comprising an effective amount of a population of the presently disclosed stem-cell-derived endodermal cells or a composition comprising said precursors in unit dosage form. In certain embodiments, the stem-cell-derived cells are mature differentiated cells, for example, insulin-secreting β cells. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering a population of the presently disclosed stem-cell-derived endodermal cells or a composition comprising thereof to a subject suffering from an endodermal disorder. The instructions can comprise information about the use of the cells or composition for treating or preventing endodermal disorder. In certain embodiments, the instructions comprise at least one of the following: description of the therapeutic agent; dosage schedule and administration for treating or preventing an endodermal disorder or symptoms thereof precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 6. EXAMPLES

The presently disclosed subject matter will be better understood by reference to the following Example, which is provided as exemplary of the invention, and not by way of limitation.

### 6.1 Example 1: Forward CRISPR Human Genetic Screen Identifies JNK Signaling as an Inhibitory Pathway of Early Endoderm Specification

### Summary

Forward genetic screens have been instrumental for understanding embryonic development and lineage decisions (Nüsslein-Volhard, 1980). Forward genetic screens have been instrumental for understanding embryonic development and lineage decisions (Nüsslein-Volhard, 1980). However, all previous screens have been performed at the organismal level, a strategy not applicable to humans. Although it is possible to use vertebrate organisms such as zebrafish and mice for genetic screens with the expectation that many though not all developmental mechanisms are conserved in humans, the throughput of such screens especially in mice has been limited for practical constraints. Furthermore, distinct regulatory mechanisms may underlie developmental control of the human genome (Chia, 2010). Human embryonic stem (ES) cells are uniquely suitable for interrogating human development and birth defects with high-throughput genetic manipulation. A genome-wide knockout screen was performed to study human development by combining the CRISPR/CAS technology with the unique property of human pluripotent stem cells (hPSCs) to self-renew while maintaining the ability to differentiate (Zhu, 2013). Such screen identifies previously known as well as novel lineage determination genes that regulate the formation of definitive endoderm (DE) cells, one of the first lineages formed in an early human embryo, which gives rise to most of the cells in respiratory and gastrointestinal organs including the lung, pancreas and liver. It was also discovered that MEKK1-MKK4/7-JNK-JUN signaling axis acts as a previously unrecognized inhibitory pathway for constraining endoderm formation. The treatment of JNK inhibitor (JNK-IN-8) improved the efficiency of ES cell differentiation to the endoderm lineage. These findings are important for creating regenerative medicine such as therapeutically relevant endoderm-derived cells, as definitive endoderm specification is the very first step toward making functional cell types in respiratory and digestive organs such as insulin-secreting β cells for type I diabetes treatment.

### INTRODUCTION

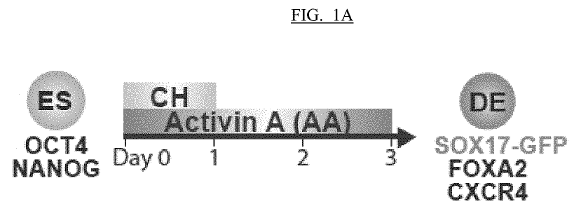

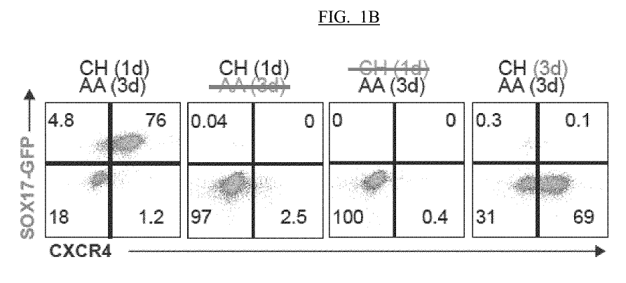

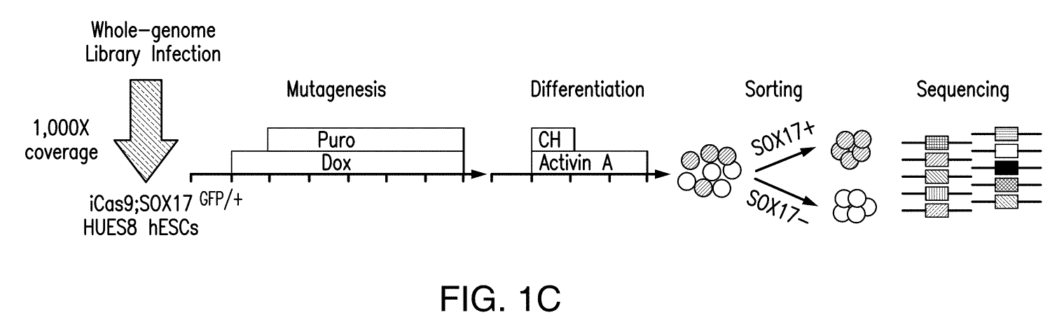

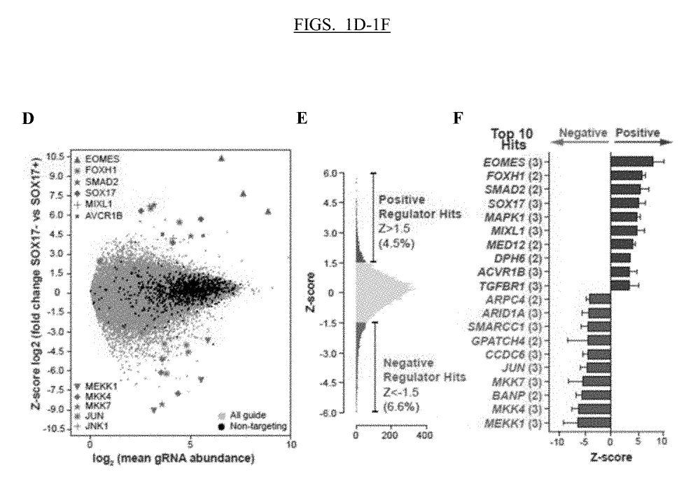

Somatic lineage specification occurs at the gastrulation stage of embryogenesis, as epiblast cells differentiate and reorganize within a narrow time window into a trilaminar structure containing ectoderm, mesoderm and definitive endoderm (DE). By utilizing mouse embryos and other model organisms, developmental biologists have uncovered requirements for discrete signaling pathways and precise spatiotemporal coordination during this highly orchestrated morphogenetic event (Zorn, 2009). These findings have largely facilitated efforts to differentiate embryonic stem (ES) cells into the three embryonic lineages and their derivatives (Zhu, 2013). For instance, high and sustained Nodal activity signals via the SMAD2-SMAD4-FOXH1 axis at the anterior primitive streak to promote the endoderm fate through a transcriptional network involving EOMES, MIXL1, GATA6/4, FOXA2 and SOX17 (Zorn, 2009). With this knowledge, it has become possible to differentiate mouse and human ES cells relatively efficiently to definitive endoderm through treatment with Activin A, a TGF-β superfamily member that mimics the activity of NodaL (D'Amour, 2005; Kubo, 2004; Tada, 2005) (FIG. 1A). However, differentiation efficiencies vary among ES and induced pluripotent stem (iPS) cell lines (Bock, 2011; Osafune, 2008), and a significant percentage of cells may fail to differentiate into endoderm suggesting the involvement of additional uncharacterized regulatory mechanisms. As both endoderm differentiation and pluripotency maintenance requires the Nodal/TGF-β pathway (Pauklin, 2015; Xu, 2008), an unknown inhibitor of endoderm gene expression has been postulated as a way to prevent precocious endoderm differentiation (Brown, 2011). Uncovering such mechanisms would facilitate the development of more robust endoderm differentiation protocols necessary for the generation of endoderm derivatives for ES/iPS-based disease modeling or cell replacement therapy. Knowledge of coordinated actions of both positive and negative developmental regulators would also advance the current understanding of how precise and robust lineage decisions are achieved and propagated during human embryonic development.

Identification of previously unknown regulators of embryonic development can be achieved through forward genetic screens in model organisms (Zhu, 2013; Anderson, 2003). Highlighting the power of forward genetics, the key endoderm regulator Nodal was itself first identified in screens performed in mice (Zhou, 1993). However, this approach cannot be directly extended to humans, posing a challenge for uncovering unique regulatory mechanisms underlying the developmental control of the human genome. An additional challenge lies in the difficulty of increasing the throughput of phenotyping mutant embryos for screens conducted in vertebrates, especially in mice due to practical constraints. Therefore, this study established a platform for high-throughput discovery of human developmental regulators by utilizing the unique property of human ES cells to self-renew while maintaining the ability to differentiate.

### Results

### A Pooled CRISPR/Cas Knockout Screen

hPSCs offer a unique model for studying human gastrulation, as in vitro human embryo culturing cannot proceed beyond this stage (Deglincerti, 2016). Somatic lineage specification occurs at the gastrulation stage of embryogenesis, as epiblast cells reorganize to a trilaminar structure containing ectoderm, mesoderm and definitive endoderm (DE). There are many known key regulators that initiate DE specification, primarily components of the Nodal/TGFβ signaling pathway and downstream transcription factors of the GATA and FOXA families, among others (Tam, 2007; Tsankov, 2015; Zorn, 2009). However, the genes that limit generation of DE are unknown, which can most effectively be investigated with a loss-of-function screen coupled to a strong phenotypic assay. Unique lineage determinant genes still remain to be found.

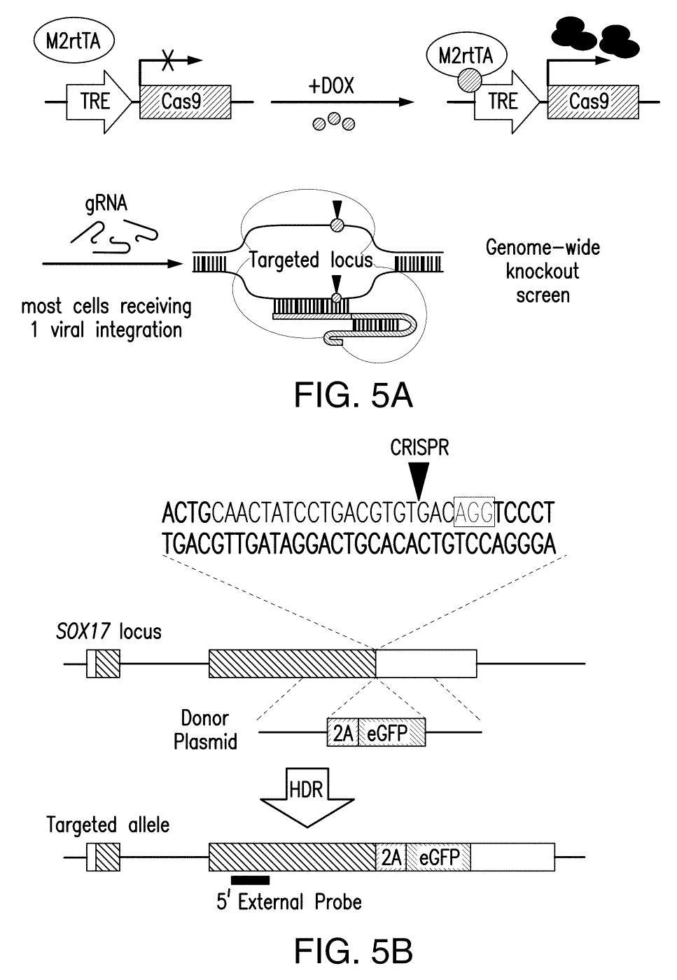

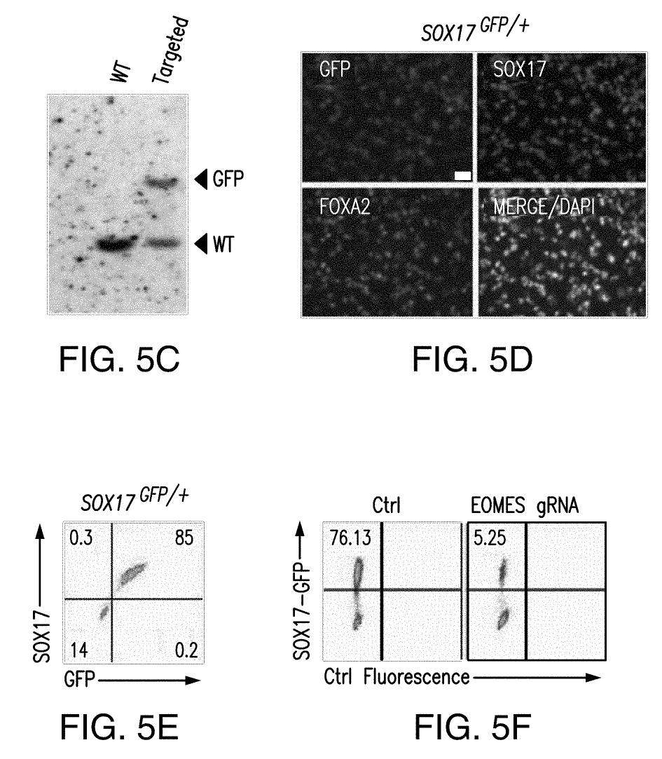

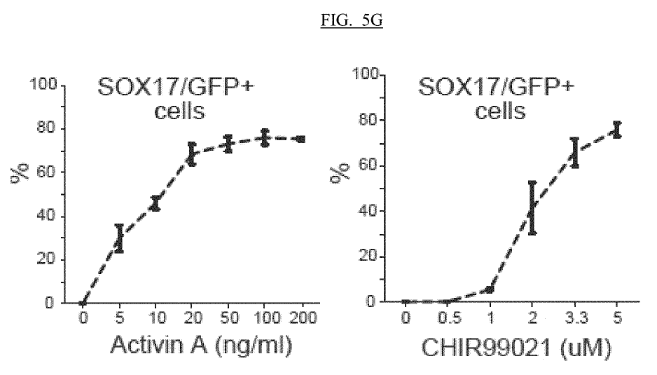

To address this, a robust CRISPR/Cas knockout screen platform was established in hPSC to identify the developmental regulators of DE formation (FIG. 5A). iCRISPR gene-editing platform established in González, 2014 and Zhu, 2015 was used, in which iCAS9 hPSCs support doxycycline inducible Cas9 expression and efficient genome editing for knockout and knockin. In particular, in iCas9 cells generated from the parental HUES8 ES cell line (González, 2014), Cas9 is integrated into the transgene safe harbor AAVS1 locus to allow doxycycline-inducible Cas9 expression, which enables efficient genome editing (González, 2014) (FIG. 5A). SOX17 is one of the best-characterized definitive endoderm markers (Wang, 2011). Utilizing a selection-free knockin strategy (Zhu, 2015), iCAS9 HUES8 hPSC line was used to generate a SOX17GFP knockin reporter line to facilitate a pooled library screen for genes that regulate the specification of the endoderm fate from hPSC (Kanai, 2002) (FIGS. 5B & 5C). Next, SOX17GFP reporter activity was validated by direct differentiating hPSC to DE under the guidance of an optimized differentiation protocol based on previous findings that activation of WNT signaling by GSK3 inhibitor CHIR-99021, and Nodal signaling by Activin A (Zhu, 2016; D'Amour, 2005) (FIG. 1A). SOX17GFP reporter faithfully reflected the endogenous SOX17 expression in DE cells (FIG. 1B), and faithful GFP reporter expression was confirmed in endoderm cells expressing SOX17 and FOXA2 by immunostaining and flow cytometry (FIGS. 5E-5F). It has been routinely generated around 65-75% endoderm cells co-expressing a panel of DE makers SOX17/FOXA2/CXCR4 as shown by both immuonstaining and flow cytometry (FIGS. 5D-5E, 1B). More importantly, these results demonstrated that the formation of SOX17+ endoderm cells required both Activin A and CHIR-99021 treatment, and the efficiency of endoderm formation was sensitive to their dosages (FIGS. 1B, 5G). both Activin A and CHIR99021 were required for inducing definitive endoderm cells co-expressing SOX17 and CXCR4, which typically constituted ˜80% of the HUES8 differentiated population (FIG. 1B). It was found that the duration of WNT activation by CHIR-99021 was critical for SOX17 expression, as prolonged CHIR-99021 treatment resulted in the formation of CXCR4+SOX17− cells, which likely resemble mesoderm cells as reported in a previous study (Tan, 2013) In order to show the proof of principle of identifying novel regulators of endoderm formation, SOX17-GFP iCAS9 line was infected with lentiviruses expressing gRNA targeting EOMES, which is a known gene necessary for the formation of definitive endoderm (Costello, 2011; Arnold, 2008; Teo, 2011). It was found that EOMES gRNA mutant line had a strong defect in endoderm formation judging by the number of SOX17/GFP+ cells compare to WT control (FIG. 5F). These experiments established a platform for an unbiased screen incorporating a reliable endoderm differentiation protocol, a GFP reporter for monitoring the differentiation at a single-cell resolution, and a positive control for comparison, and supported the feasibility of a pooled based CRISPR knockout screen.

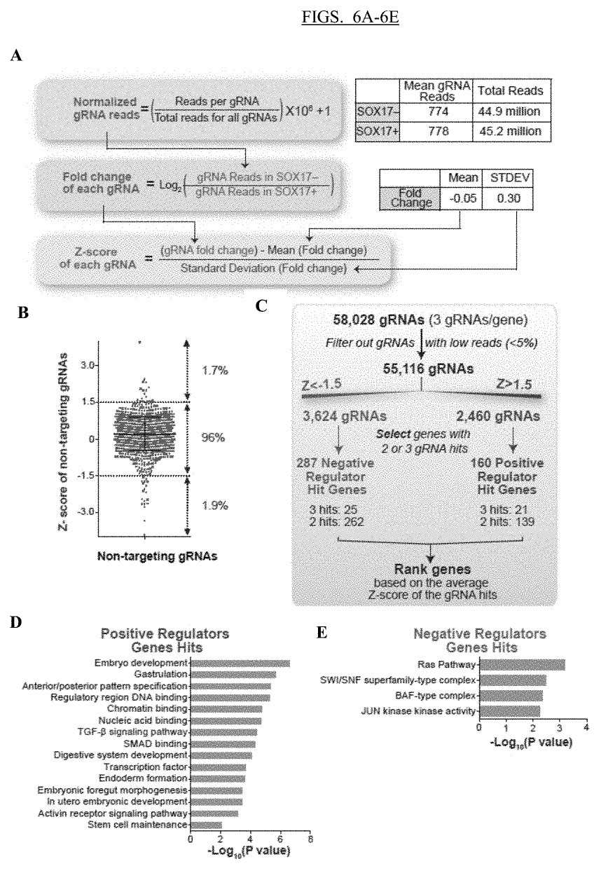

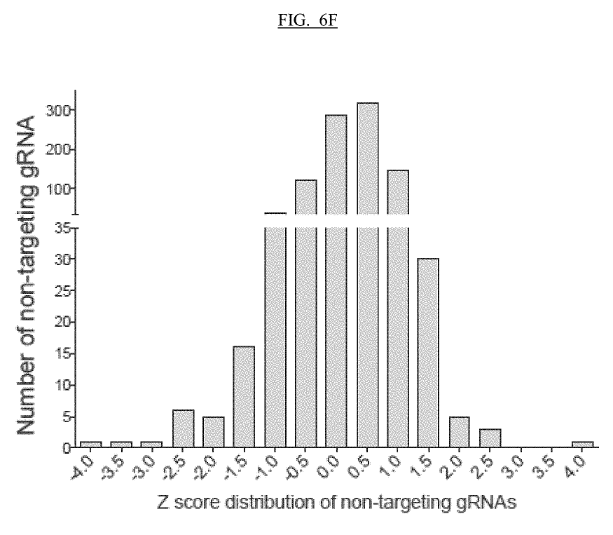

Two hundred million iCas9 SOX17^{GFP/+} cells were infected at a low multiplicity of infection (MO1) of 0.36 (estimated to give a 1,000-fold coverage) with the pooled lentiviral human GECKOb v2 library (Sanjana, 2014). The GECKOb library contains 57,028 gRNAs targeting ˜19,009 human genes (3gRNAs per gene) and 1,000 non-targeting gRNA controls. After six days of doxycycline treatment to induce Cas9 expression followed by three days of differentiation, the mutated hESC pool was differentiated to endoderm fate, and SOX17/GFP+ endoderm and SOX17/GFP− non-endoderm cells were isolated through fluorescence-activated cell sorting (FACS) (FIG. 1C). The abundance of individual gRNAs in each population was determined by high-throughput sequencing: gRNAs that target genes that either promote or inhibit the formation of endoderm should be depleted or enriched, respectively, in SOX17/GFP+ cells compared to SOX17/GFP− cells. Z score was calculated for each gRNA based on the ratio of gRNA reads in the SOX17/GFP− versus SOX17/GFP+ population (FIG. 6A).

The results showed that 96% non-targeting controls had a Z score between −1.5 and 1.5, indicating relatively low noise (FIGS. 6B, 6F). This screen successfully recovered multiple gRNAs targeting EOMES, SOX1 7, and Nodal/WNT pathway regulators such as SMAD2, FOXH1 and CTNNB1 depleted in the SOX17/GFP+ cells (FIG. 1D). In order to systemically rank the gRNAs to a gene level, gRNAs with low reads were first filtered out, as results from low abundance gRNAs tend to be unreliable (Parnas, 2015). Next, gRNAs with Z score greater than 1.5 (positive regulators), and gRNAs with Z score less than −1.5 (negative regulator) were analyzed (FIGS. 6C, 1E). A gene was considered a hit when at least 2 gRNAs or 3 gRNAs made the cutoff, and the average Z-score of these gRNAs was used to rank individual hits (FIG. 6C). This step-wise filter leaded to a list of 160 positive and 287 negative regulator hit genes (FIG. 1E). Gene ontology analysis of the 160 positive regulator hits showed enrichment of genes involved in the TGF-β pathway, gastrulation, and endoderm formation; whereas the 287 negative regulator hits were enriched for genes in the SWI/SNF complex, RAS pathway, and c-Jun N-terminal protein kinase (JNK) pathway (FIGS. 6D-6E). The hit list was further ranked based on the average Z score of the gRNAs for each hit gene (FIG. 7). Using this method, many known endoderm positive regulators were found passed through the filter and ranked highly in the top 50 positive regulators list with Z-scores higher than 3. For example, top 10 positive and negative regulators are enriched for 3gRNA hits and with high absolute value of Z-score, and notably, the top 10 positive regulator hits included almost all non-redundant, cell-autonomously required genes in the Nodal pathway (ACVR1B, SMAD2, SMAD4 and FOXH1) (Robertson, 2014) as well as established endoderm transcription factors (EOMES and MIXL1) (Zorn, 2009) (FIGS. 1D, 1F), supporting the comprehensive identification of known endoderm regulators. Gene ontology analysis showed TGF-b pathway, gastrulation, and endoderm formation genes are enriched in the positive regulators. Among the negative regulators, it was observed that JNK pathways member were ranked in top 5 (FIG. 1F) and gene ontology analysis also showed SWI/SNF complex (SMARCC1 and ARID1A) were also enriched in this group (FIGS. 6D, 6E).

### Validation of Screening Hits

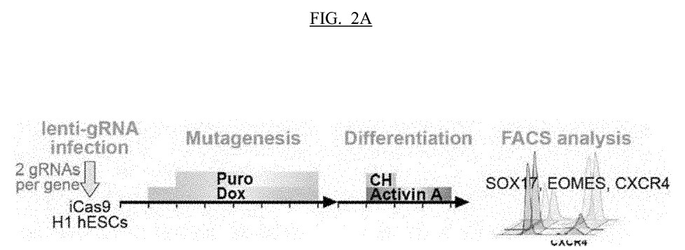

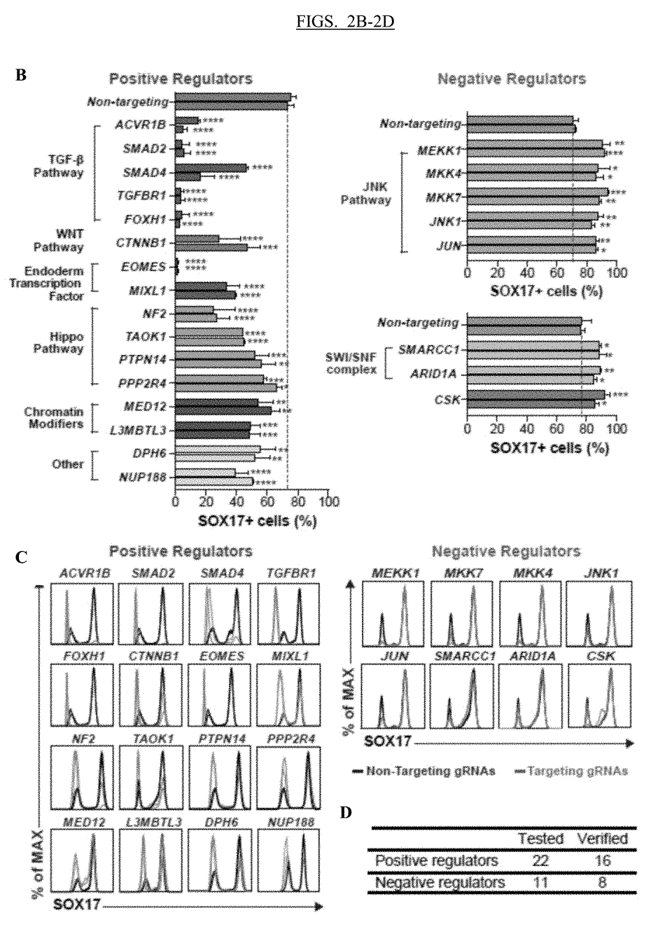

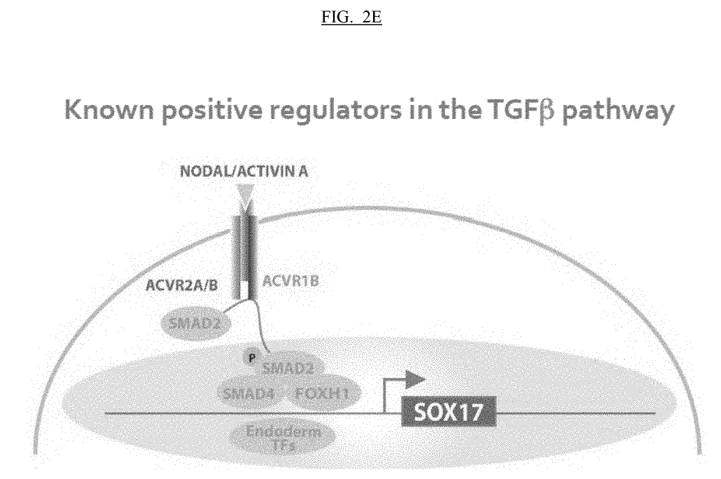

The validation experiments paralleled the screening procedure with some modifications. Top hit genes were validated by using the lenti-CRISPR KO approach (FIG. 2A). For each candidate, 2 gRNAs were used along with 2 non-targeting gRNA controls in individual differentiation assays that mirror the pooled screening strategy (FIG. 2A). The H1 ES cell line was used, instead of HUES8, to exclude genes with background or line-specific effects (FIG. 2A). Using H1 iCas9 cells (Shi, 2017), two gRNAs were against each candidate along with two non-targeting gRNA controls in individual, rather than pooled differentiation assays. The effect on DE formation was evaluated based on directly measurement of intracellular SOX17 staining (instead of relying on a SOX17GFP reporter) and additional endoderm markers EOMES and CXCR4. A total of 24 positive regulators and 11 negative regulators were verified, and 7 of validated hits are false positive hits. Overally, 24 of the 33 hits tested were verified (FIGS. 2B-2D, FIG. 7)

The 16 verified positive regulators included key genes of Noda and WNT signaling pathways that were necessary for definitive endoderm differentiation. This finding was previous work reporting gastrulation defects in Acvr1b, Smad2, Smad4, Foxh1, Ctnnb1 knockout mice²²⁻²⁶ (Gu, 1999; Nomura, 1998; Sirard, 1998; Yamamoto, 2001; Haegel, 1995; Schier, 2003; van Amerongen, 2006). Interestingly, it was also confirmed that TGFBR1 is also required for human definitive endoderm formation, however, TGFBR1 KO mice does not exhibit any obvious gastrulation phonotype (Larsson, 2001). Definitive endoderm specific transcription factors are another important players in orchestrating the lineage specification program. Here, it was showed that EOMES and MIXL1 are essential for formation of human SOX17+ endoderm cell, which was consistent with previously demonstrated roles of key definitive endoderm transcription factors in mice (Arnold, 2008; Hart, 2002). However, MIXL1 KO cells have similar number of EOMES+ cell compared to control, unlike SMAD2/SMAD4/FOXH1/CTNNB1/TGFBR1 KO cells with very low number of EOMES+ cells, which suggests that TGFβ/WNT pathways regulate EOMES expression and EOMES is upstream of MIXL1. The recent work also demonstrate GATA6 is an important regulator for efficient definitive endoderm formation, and GATA6/GATA4 double KO cells have a severer DE differentiation phenotype. The Z score of GATA6/4 from the screen result is associated with the degree of phenotype that were previously observed.

Four upstream Hippo pathway genes (NF2, TAOK1, PTPN14, PPP2R4) (PlouffeHart, 2016; Ribeiro, 2010) are also enriched in the top 50 positive regulators. Their primary function, although acting on different mechanisms, is to negatively regulate YAP1 nuclear activity (Johnson, 2014). The exact role of YAP1 or other Hippo pathway members in the formation of definitive endoderm cess is less clear, however, transient siRNA knocking down YAP1 can lead to a ˜3-fold up-regulation of mesendoderm genes MIXL1, EOMES and T (Beyer, 2013; Estarás, 2015). Interestingly, YAP1 is recovered from the negative regulators list with a Z-score of −2.9 (Ranked 49th). The genetic analysis of upstream hippo genes provides the first clue that hippo signaling activation is also important for efficient DE differentiation. In addition, some less well-studied new gene involved with DE differentiation were also identified, such as such as MED12 (Mediator Complex Subunit 12), L3MBTL3 (a polycomb group protein), DPH6 (a diphthamide biosynthesis enzyme required for the modification of the eukaryotic translation elongation factor EEF2) and NUP188 (a nuclear pore complex protein) (FIGS. 2B-2C, 7). MED12 is important for cell-type specific DNA looping (Kagey, 2010). Mutation of MED12 are associated with x-linked dominant mental retardation, Lujan-Fryns syndrome and FG syndrome (Wang, 2013). L3MBLT3 is a member of MBT domain protein found in polycomb group and L3MBTL3 knockout mice have impaired maturation of myeloid progenitors and are embryonic lethal (Arai, 2005). DPH6 is responsible for the diphthamide modification on eukaryotic translation elongation factors 2 (eEF2), and mice unable to complete the diphthamide biogenesis process are embryonic lethal (Uthman, 2013).

Prior to this study, there was little knowledge about the negative regulators of endoderm differentiation. Notably, in the negative regulator category, five of the validated negative regulators were key members of the JUN N-terminal Kinase (JNK) pathway genes. JNK is a subfamily of the mitogen-activated protein kinase (MAPK) superfamily, and the pathway is activated by a variety of environmental signals including stress, cytokines and growth factor (Davis, 2000). However, a role of the JNK pathway in regulating endoderm differentiation has not been previously reported. The validation experiment identified MAPK kinase kinase MEKK1 (MAP3K1, rank 1), MAPK kinase MKK4 (MAP2K4, rank 2) and MKK7 (MAP2K7, rank 4), JNK1 (one of the three JNK genes, rank 12), and the JNK substrate transcription factor JUN (C-JUN, rank 5) as negative regulators of hPSC differentiation into DE (FIG. 1F). Overall, the validation of ˜70% of the top hits examined (FIGS. 2D & 7) supports with high confidence the positive and negative hits identified from this genome-wide screen for endoderm regulators.

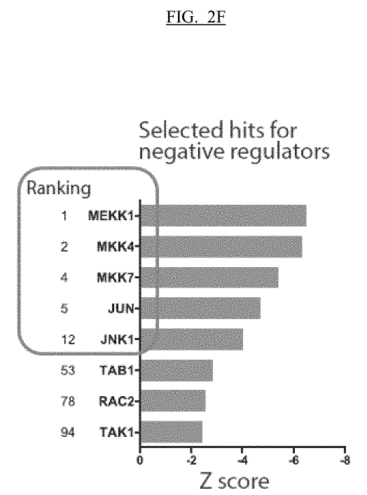

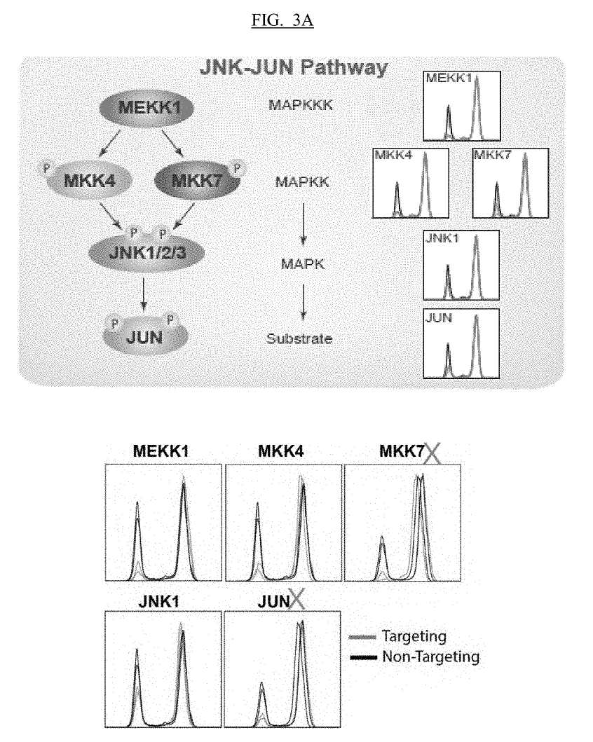

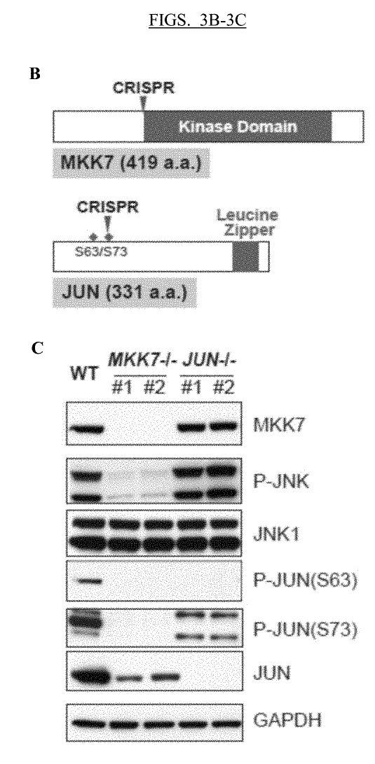

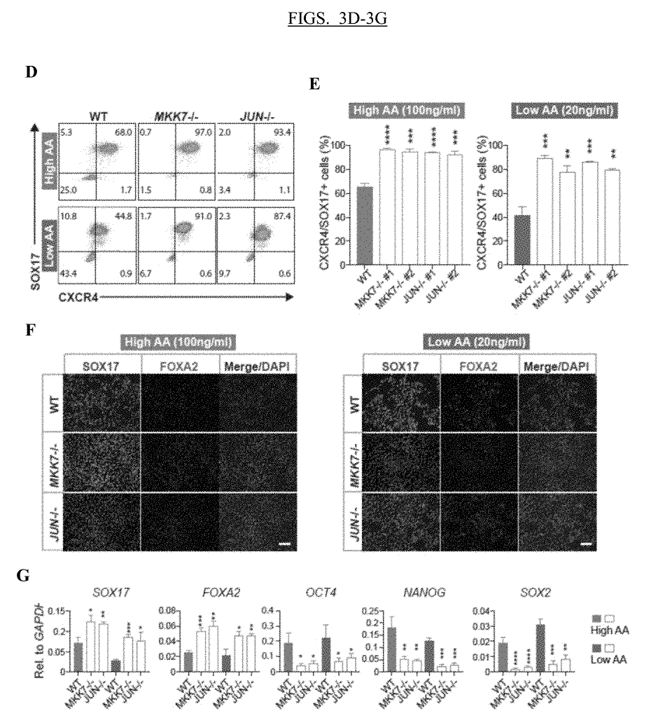

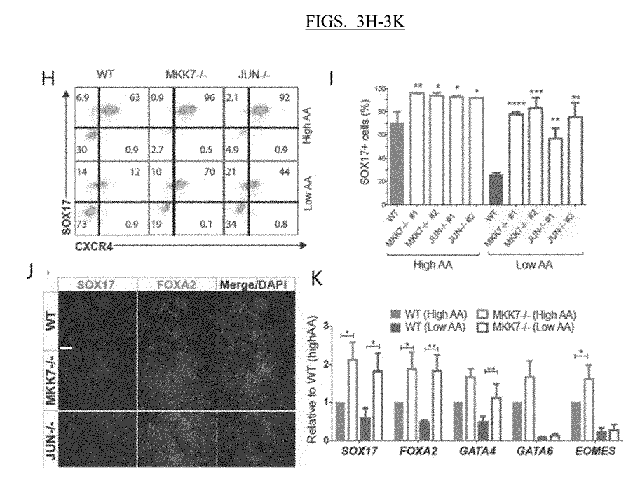

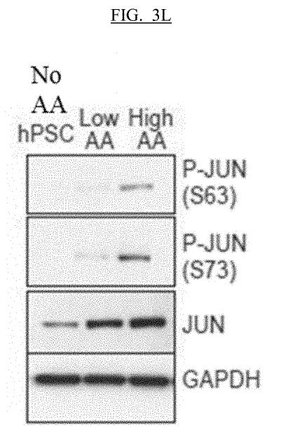

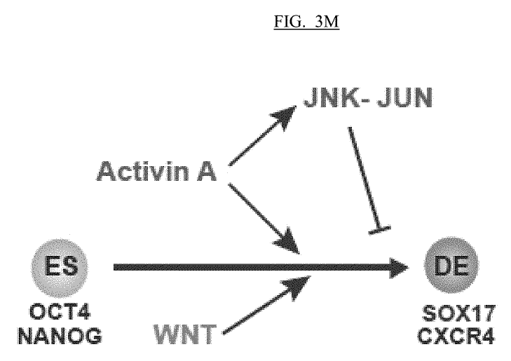

JNK pathway inhibits endoderm differentiation Although each of the five JNK pathway genes had been verified individually using gene-specific gRNAs expressed from lentiviral vectors (FIGS. 2B-2C), those experiments were performed on non-clonal populations in which some cells could retain gene activity, e.g., due to in-frame mutations. Thus, to further investigate on how the JNK pathway inhibits endoderm formation (FIG. 3A), clonal MKK7 and JUN homozygous knockout (KO) hPSC lines (two lines each) in the H1 background (FIGS. 3B & 9A) were generated. Western blotting results verified the absence of MKK7 and JUN proteins in the corresponding KO cells after differentiation to endoderm (FIGS. 3C & 9A). Consistent with the known JNK signaling cascade, knocking out MKK7 strongly reduced the phosphorylation level of JNK, and the phosphorylation level of JUN became undetectable in MKK7 KO hPSC lines (FIG. 3C).

After induction of DE differentiation for 3 days, MKK7 and JUN KO cells readily formed DE cells co-expressing SOX17, CXCR4 and EOMES at a higher efficiency (>90%) compared to wild-type H1 cells (˜70%) in response to the typical 100 ng/ml Activin A treatment used for endoderm induction (FIG. 3D). When treated with a reduced Activin A dosage (5 ng/ml), only a small number of wild-type cells expressed endoderm markers, yet MKK7 and JUN KO cells still expressed a higher percentage of endoderm markers SOX17, CXCR4 and FOXA2 as detected by flow cytometry and immunostaining (FIGS. 3H-3J).

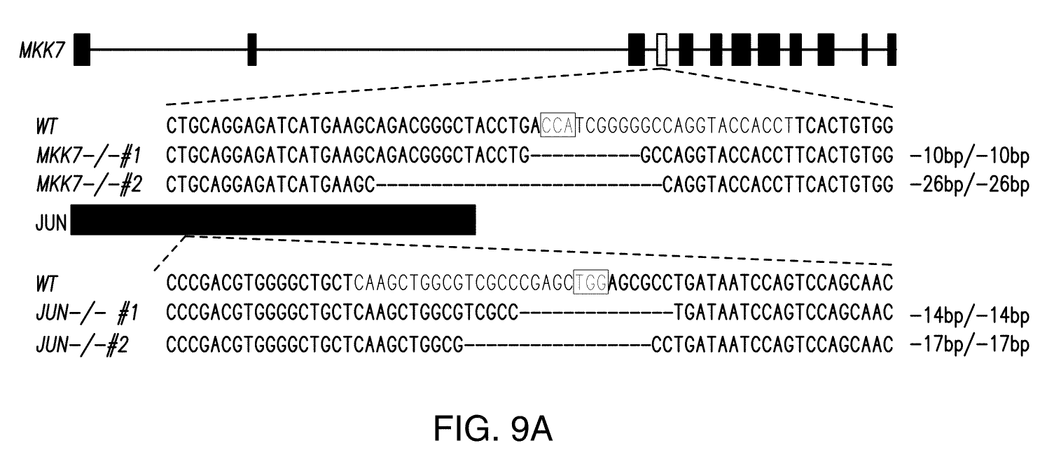

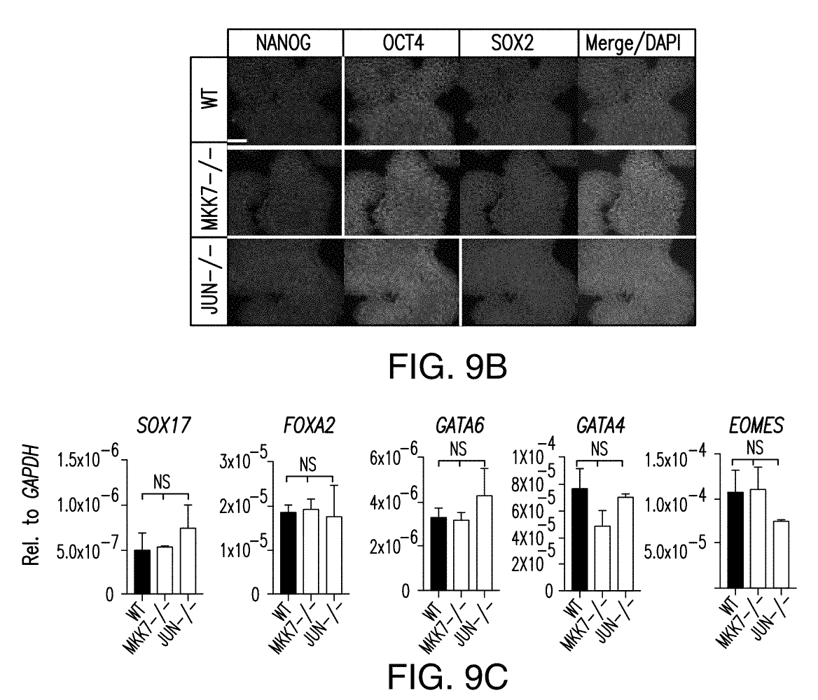

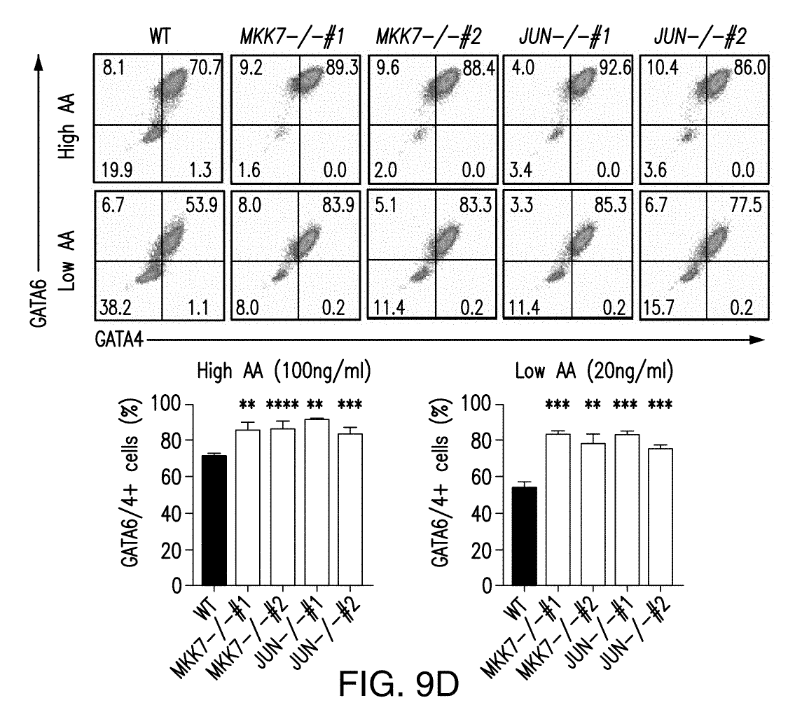

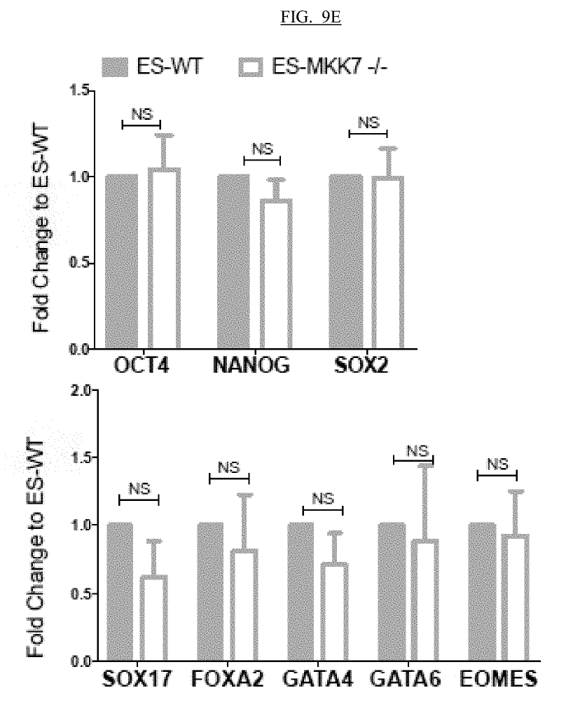

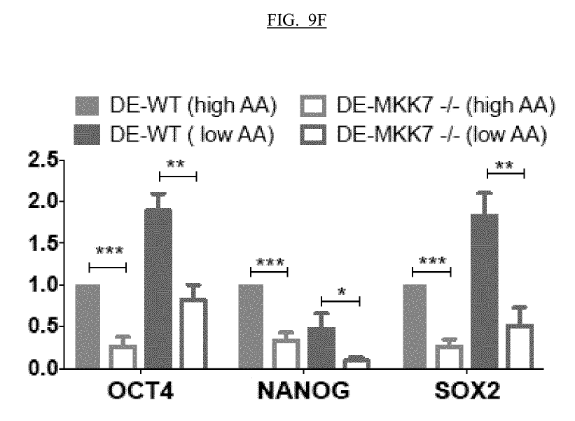

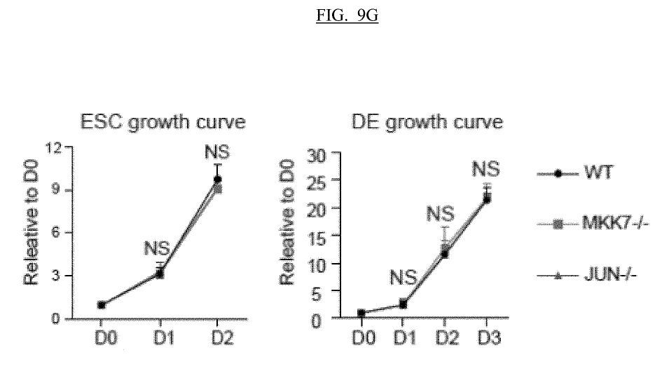

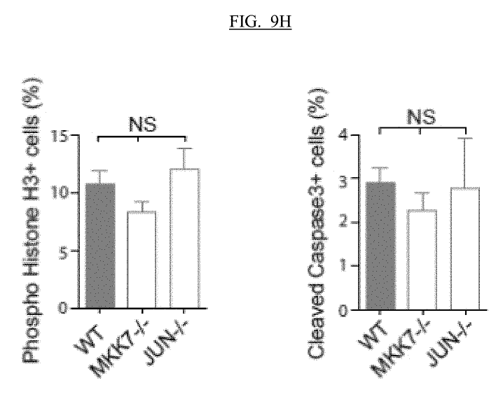

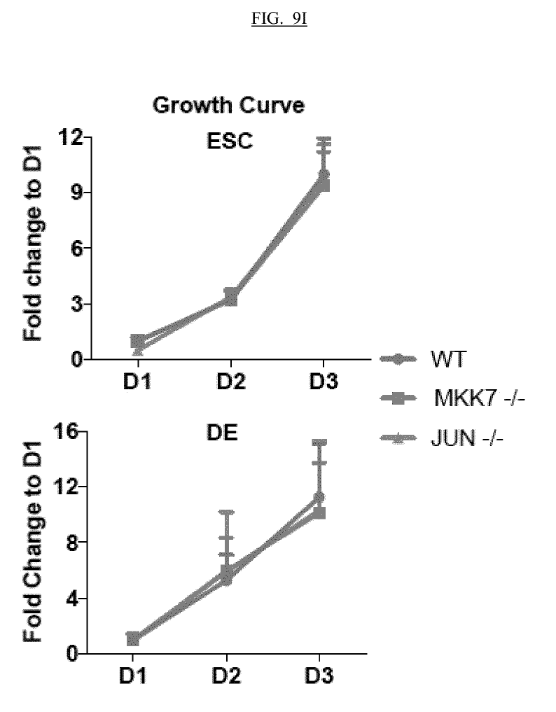

When treated with a different dosage of low Actin A (20 ng/ml), similar results were observed; a reduced percentage of cells in the WT background, but MKK7 and JUN KO cells still formed ˜90% endoderm cells co-expressing SOX17 and CXCR4 as detected by flow cytometry (FIGS. 3D-3E). Similar results were observed for endoderm markers GATA6 and GATA4 by flow cytometry with high (100 ng/ml) and low Activin A (20 ng/ml) conditions (FIG. 9D). Immunostaining and RT-qPCR analysis confirmed up-regulation of endoderm marker genes (SOX17 and FOXA2) accompanied by the further down-regulation of pluripotency genes (OCT4, NANOG and SOX2) in MKK7 and JUNKO cells compared to WT controls (FIGS. 3F, 3G, 3K, 9F). No significant difference was observed in proliferation and apoptosis of MKK7 and JUN KO cells compared to wild-type control (FIGS. 9I-9J). These findings suggest that inactivation of JNK pathway could unleash the DE differentiation potential and result in a purer DE population. Genetic inactivation of JNK-JUN pathway also increases the sensitivity of the cells response to Activin A treatment.

These phenotypes were not due to a role of MKK7 or JUN in maintaining the pluripotent state, as no gene expression changes of pluripotency and differentiation related genes were observed in KO versus WT ES cells before differentiation (FIGS. 9B-9C). MKK7 and JUN KO cells have normal pluripotent stem cell morphology FIG. 9B). qPCR analysis also confirmed that pluripotency markers (OCT4, NANOG, SOX2) and definitive endoderm gene markers are not altered at the pluripotency stage (FIG. 9E). MKK7 or JUNKO cells did not exhibit a difference in cell growth during ES cell self-renewal or endoderm differentiation (Extended Data FIG. 9G), nor did flow cytometry detect a significant difference in the number of cells expressing proliferation or apoptosis markers (phospho-histone H3 and cleaved caspase-3, respectively) during DE differentiation (FIG. 9H). Interestingly, level of phosphorylation of JUN at the pluripotency stage was not detected, but the level of JUN phosphorylation was associated with the dosage of Activin A during DE differentiation (FIG. 3L). Therefore, inactivation of JNK pathway unlikely promoted endoderm differentiation at the pluripotency stage.

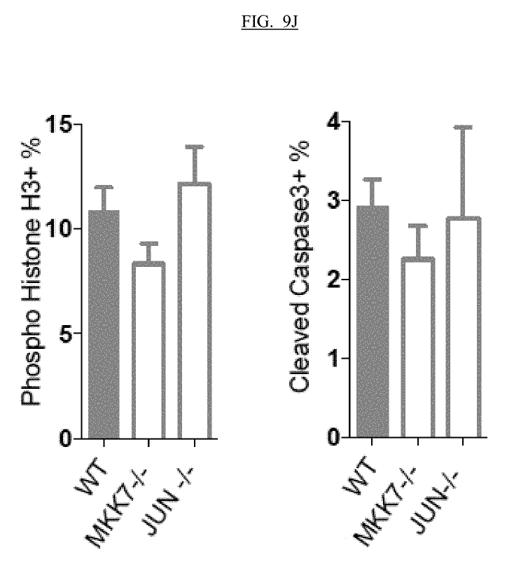

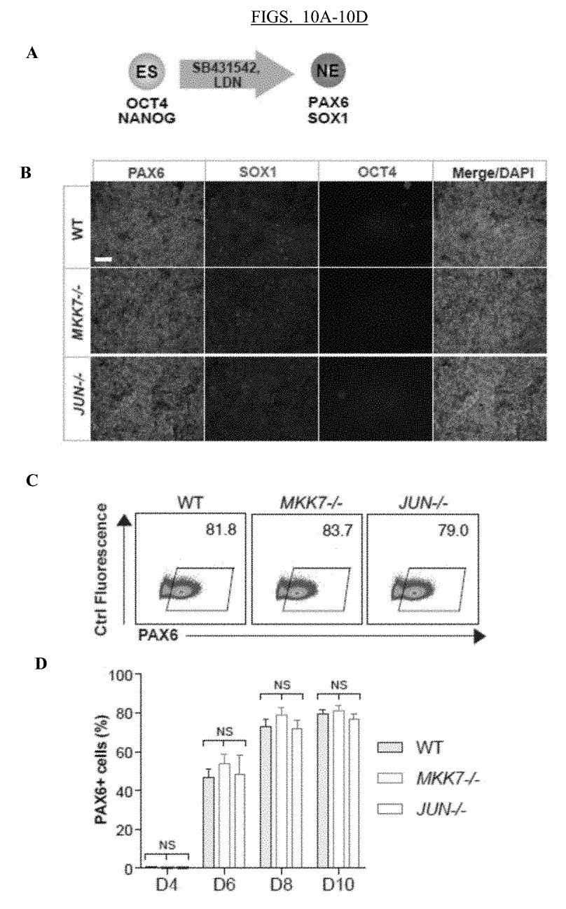

It was also found that the JNK pathway did not inhibit the differentiation to all lineages. It was examined whether MKK7 and JUN KO hPSCs also exhibit phenotypes in differentiation to the neuroectoderm (NE) lineage using the dual-SMAD inhibition protocol (Chambers, 2009) (FIG. 9A). MKK7 and JUN KO ES cells exhibited no difference compared to WT cells in the efficiency or kinetics of forming neuroectoderm cells expressing PAX6 and SOX1 after 10 days of differentiation as determined by immunostaining and flow cytometry (FIGS. 10A-10D). No phenotype was observed at earlier differentiation time points based on the quantification of PAX6 positive cells by flow cytometry. Time-course flow cytometry analysis during NE differentiation also showed no change in terms of PAX6 positive cells percentile between WT cells and MKK7/JUN KO cells (FIGS. 10B-10C). Thus the JNK pathway inhibits the differentiation of ES cells to the endoderm lineage specifically, and does not have a general impact on the dissolution of the pluripotency state.

### JNK Inhibitor and Nodal Signaling

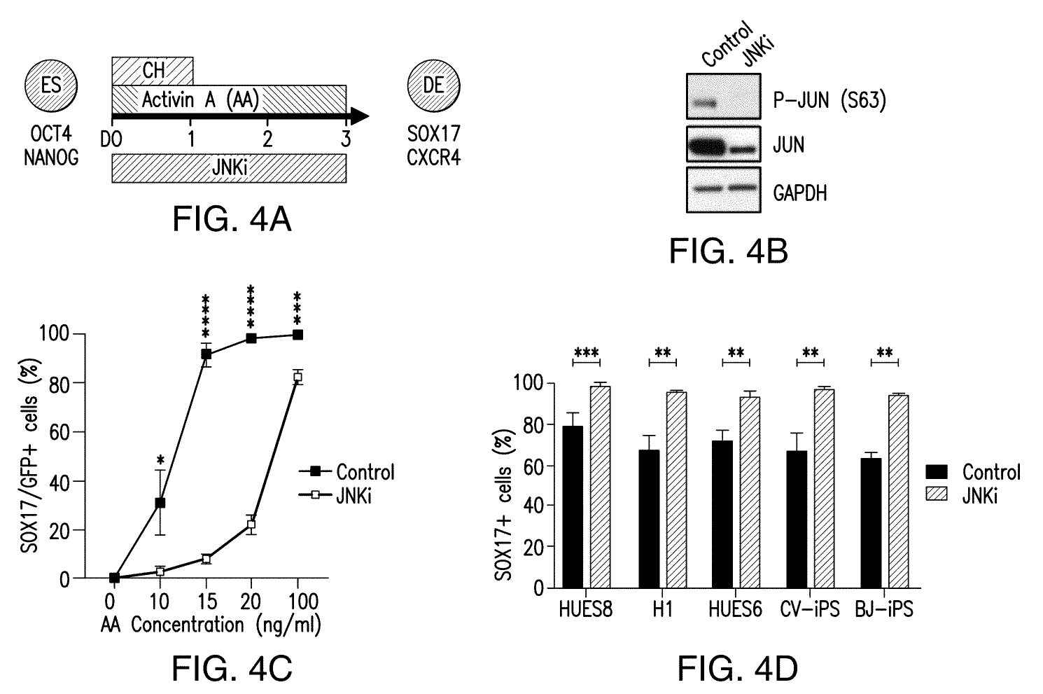

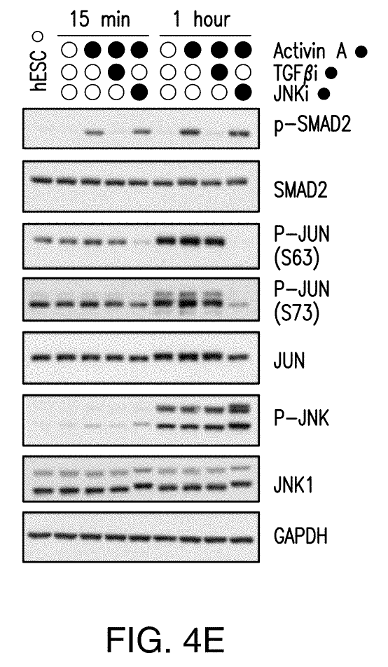

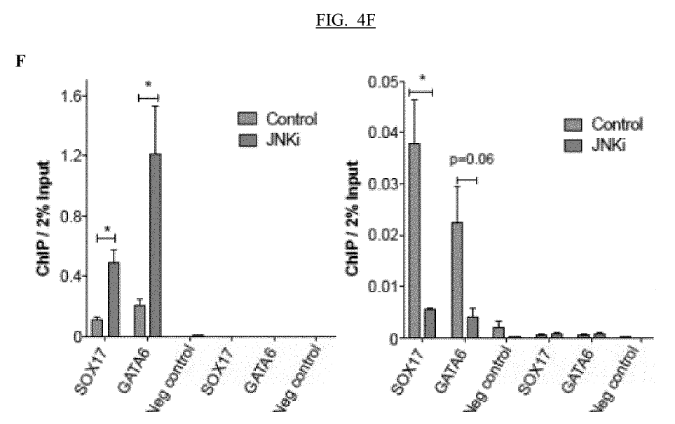

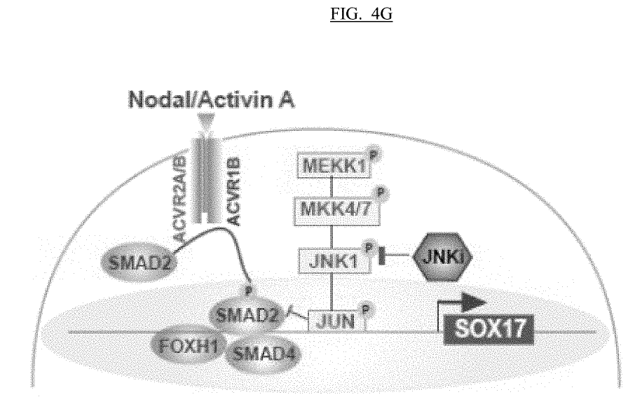

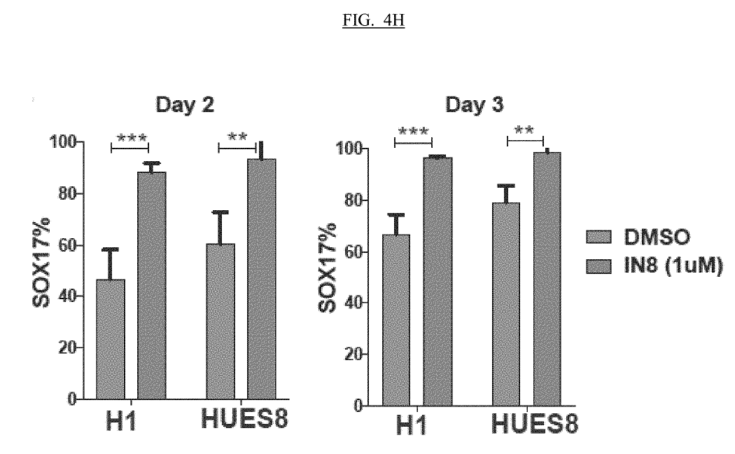

The role of JNK signaling constraining endoderm differentiation provides the logic that JNK pathway could be therapeutically explored to promote endoderm differentiation by applying small molecule JNK inhibitor during definitive endoderm differentiation (FIG. 3M). After screening a collection of commercially available JNK inhibitors, JNK-IN-8 was used in the current study due to its high specificity and potency (Zhang, 2012) (FIG. 6A). Because JUN is not phosphorylated at the hPSC stage, JNK-IN-8 was added during the process of DE differentiation (FIG. 4A). Using two different hPSC background (H1 and HUES8-SOX17GFP), it was shown that the 1 μm JNK-IN-8 strongly promoted DE differentiation (˜96% in H1, ˜98% in HUES8), compared to DMSO treated control (˜66% in H1, and ˜79% in HUES8) in 3 days, and this effect of JNK-IN-8 is stronger at Day 2 (FIG. 4H). Western blot analysis confirmed that phosphorylation of JUN became undetectable in JNK-IN-8 treated cells and total JUN level was also down-regulated as expected in cells with reduced JUN phosphorylation (Massague, 2012) (FIG. 4B).

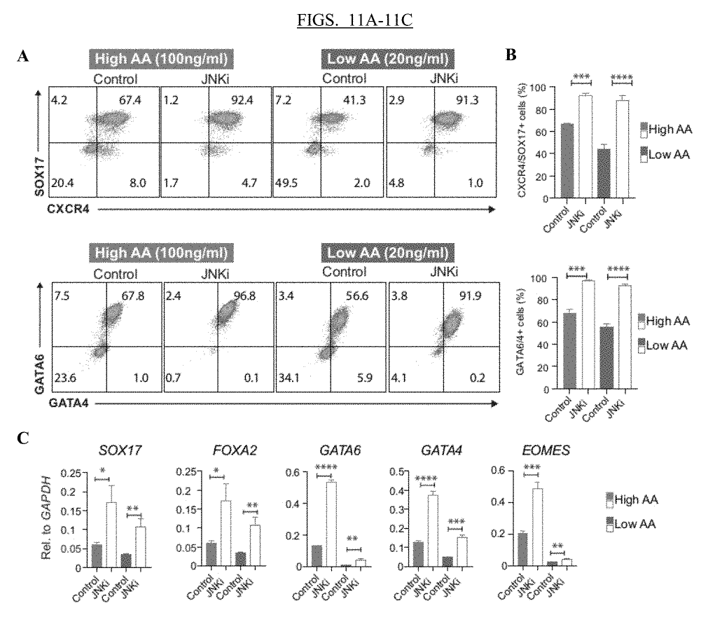

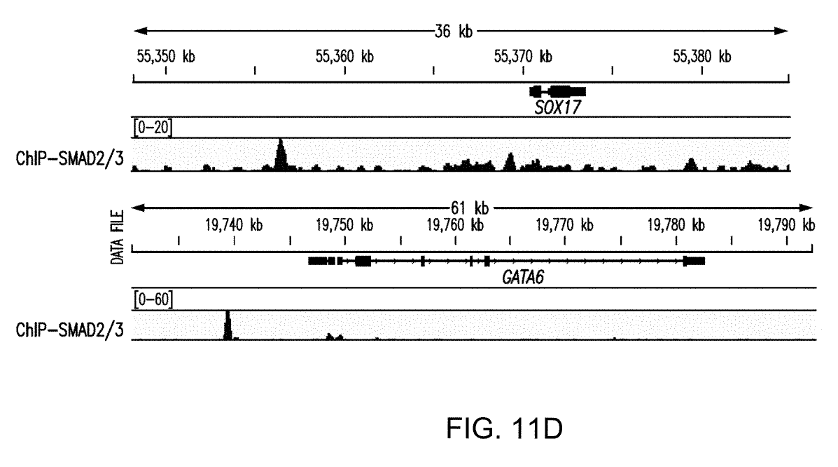

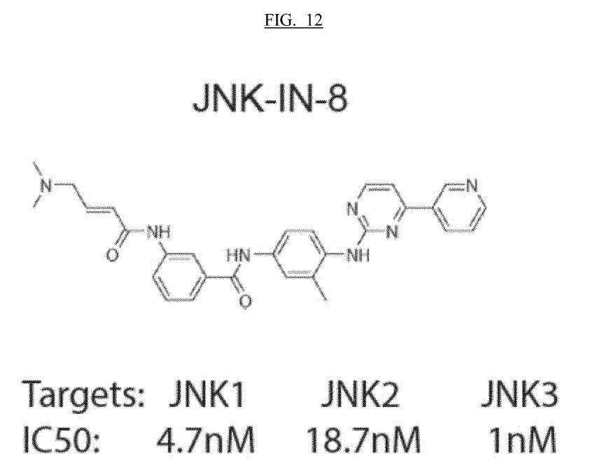

Similar to phenotypes observed in MKK7 and JUNKO cells, JNK-IN-8 treatment improved the efficiency of endoderm differentiation to greater than 90% based on flow cytometry and increased levels of endoderm gene expression from RT-qPCR analysis in either high or low Activin A condition (FIGS. 11A-11C). Activin A dose titration experiments on HUES8 SOX17^{GFP/+} cells (FIG. 4C) showed that JNK-IN-8 treatment did not omit the requirement for Activin A, but it promoted efficient induction of SOX17-expressing endoderm with a much lower Activin A dose: ˜95% SOX17+ cells formed after treatment with 20 ng/ml Activin A. In addition to H1 and HUES8 cells, JNK-IN-8 also significantly improved endoderm differentiation efficiency from HUES6 ES cells, and BJ and CV iPS cells, which in the absence of the inhibitor varied in differentiation efficiency between 60-80% (FIG. 4D). These findings demonstrate that JNK inhibition improves endoderm differentiation efficiency and reduces differentiation variability among different ES and iPS lines.

Since JNK inhibition does not bypass the requirement for Activin A/Nodal signaling, it was hypothesized that JNK inhibition may enhance endoderm differentiation through increasing SMAD2 phosphorylation or promoting its transcriptional activity³⁶. Western blotting analysis at 15 minutes and 1 hour after initiating differentiation showed that Activin A treatment induces C-terminal SMAD2 phosphorylation (at Ser465/467) as expected, an effect blocked by SB431542, a selective inhibitor of ACVR1B/ALK4, TGFBR1/ALK5 and ACVR1C/ALK7 (FIG. 4E). It was verified that JNK-IN-8 treatment inhibited JUN phosphorylation, however, this did not change the level of C-terminal SMAD2 phosphorylation. Conversely SB431542 treatment also did not affect the level of JUN phosphorylation. Thus, inhibition of JNK pathway enhances endoderm differentiation through an Activin A/Nodal-dependent mechanism that does not involve the regulation of SMAD2 phosphorylation, but possibly by inhibiting SMAD2 binding to its transcriptional targets. To investigate the latter scenario, ChIP-qPCR assays were performed and confirmed previously reported” binding of SMAD2/3 to enhancer regions of SOX17 and GATA6 during endoderm differentiation but not to a negative control region upstream of the SOX17 promoter (FIG. 4F and FIG. 11D). Notably, significant binding of JUN to these same regions occupied by SMAD2/3 was detected. Furthermore, treatment of JNK-IN-8 not only diminished the binding of JUN, confirming the specificity of the JUN-ChIP results, but also enhanced the binding of SMAD2/3 to the SOX17 and GATA6 enhancers. Collectively, these results suggest that JUN and SMAD2/3 compete for binding to endoderm target genes, and inhibition of the JNK pathway enhances SMAD2/3 binding, thus increasing transcription of endoderm target genes and promoting the induction of endoderm fate. The instant study has completed the first human genetic screen to identify novel positive and negative regulators of definitive endoderm. Out results suggest that definitive endoderm lineage commitment is specified by lineage inductive signalings and constrained by inhibitory signalings. It has been shown that the dosage requirement of WNT and Nodal signaling is essential for DE differentiation and hPSCs need a collection of transcription factors, epigenetic regulators, and signaling transduction molecules to enable proper differentiation to occur. Genes identified from this screen are potential great resources for understanding human embryonic development and congenital birth disorder. This screen also discovers the unexpected role of JNK pathway in inhibiting human definitive endoderm differentiation. The immediate application of this finding is that the use of JNK inhibitor (JNK-IN-8) as a simple tool to improve human definitive endoderm differentiation. Taken together, it has been demonstrated that the current forward human genetic screen platform offers a new experimental paradigm for understating human development. With new generation of CRISPR screen library targeting enhancer, such strategy can be also used to interrogate the role of non-coding DNA sequence during human development (Canver, 2015).

### Discussion

CRISPR/Cas-mediated gene knockout, repression and activation methods have greatly accelerated genetic screens in cellular and animal models (Parnas, 2015; Shalem, 2014). Most large-scale screens thus far introduced pooled libraries into cancer cell lines for cell death/survival or proliferation associated phenotypes. No previous CRISPR screens were performed to study lineage commitment during development. Previous screens in mouse and human ES/iPS cells have centered on the maintenance or dissolution of the self-renewing pluripotent state, relying primarily on RNA interference (RNAi) based methods (Chia, 2010; Gonzales, 2015; Gonzalez, 2016). In order to perform a genome-wide genetic perturbation screen to identify human developmental regulators for the first time, the current study utilized an established endoderm differentiation platform, and created a knockin GFP reporter cell line to monitor endoderm lineage commitment at cellular resolution. Considering the complexity of monitoring lineage commitment during differentiation compared to assays based on cell survival or proliferation, the current study chose maximize the sensitivity of the screen by maintaining a relatively high 1,000-fold coverage of the library throughout the screening process. The screen identified many of the previously known regulators such as Nodal pathway components and uncovered previously unknown genes including those encoding transcription factors, epigenetic regulators, and signaling transduction modulators, which together provide a more complete understanding of endoderm differentiation.

During gastrulation, Nodal signals via the SMAD2-SMAD4-FOXH1 axis to promote endoderm formation, whereas the secreted proteins Lefty1, Lefty2, and Cer1 (Cerberus 1) act as Nodal antagonists to restrict endoderm or mesendoderm formation in mice (Meno, 1999; Perea-Gomez, 2002). However, the only known cell-autonomous negative regulator during mammalian gastrulation is the general transcription factor Drap1. Drap1 knockout mouse embryos show phenotypes similar to Lefty2 mutants, and in vitro assays suggest that physical interaction between Drap1 and Foxh1 inhibits the binding of Foxh 1 to the Nodal-response element (Iratni, 2002). The identification of the unexpected inhibitory role of MEKK1-MKK4/7-JNK-JUN signaling axis in endoderm differentiation highlights the power of this genome-wide CRISPR screen to identify new genes and entire pathways. Negative regulation of Nodal signaling is important for establishing a Nodal gradient and setting a morphogen boundary that ensures the spatiotemporal precision and robustness of developmental programs. Additional negative regulators could be identified from the screen (FIG. 7) interact with the JNK pathway or act in parallel during endoderm differentiation.

Developmental pathways are often dysregulated in cancer cells and other biological contexts, and JNK activity has been shown to inhibit TGF-β signaling in a number of studies (Javelaud, 2007). For instance, repression of TGF-β signaling caused by hyperactivation of the JNK pathway contributes to HTLV-1 associated adult T-cell leukemia. In this context, phosphorylated JUN interacts with Smad3 and inhibits Smad3 DNA binding activity (Arnulf, 2002). Studies performed in COS-7 and HepG2 cell lines show that JUN also suppresses SMAD2 transcriptional activity by stabilizing a SMAD2 co-repressor complex with SKI or TGIF (Pessah, 2002; Pessah, 2001). Adding to these known regulatory mechanisms, the current study discovered that JUN and SMAD2/3 compete for binding at the enhancers of the Nodal target genes, thus fine-tuning the output of Nodal signaling during endoderm formation. Based on the efficacy of a small molecule JNK inhibitor, the current findings may be further exploited in cancer therapeutics for targeting the JNK and TGF-β pathways.

Overall, the current findings support the broader utility of genetic screens in human ES cells for uncovering developmental regulators. In addition the identification of druggable genes, such as JNK1, could be useful in improving ES cell differentiation for regenerative medicine. With newer generations of CRISPR libraries, this screening platform could be utilized to identify novel enhancers or non-coding RNAs in endoderm differentiation (Korkmaz, 2016; Liu, 2017). While the current screen focused on one of the earliest lineage decisions during development, future screens may identify genes that regulate later differentiation events such as the formation of cardiomyocytes or pancreatic β cells for understanding mechanisms underlying congenital heart disease or neonatal diabetes. Moreover, unlike in mice, there is no allelic segregation in the cell-based system, thus opening the door to more complex screens such as screens for disease modifiers in a sensitized genetic background.

### Methods

### Human ES/iPS Cell Culture

This study used three human ES cell lines: HUES6, HUES8 and H1 (NIH approval number NIHhESC-09-0019, NIHhESC-09-0021 and NIHhESC-10-0043); and two human iPS cell lines (BJ and CV iPS cell lines). The generation of the iCas9 lines from HUES8 and H1 cells was previously described (González, 2014; Shi, 2017). Undifferentiated human ES and iPS cells were routinely maintained as previously described (Shi, 2017) in the chemically defined feeder-free E8 condition (Thermo Fisher Scientific, A1517001) at 37° C. with 5% CO2, and routinly confirmed to be mycoplasma-free by the MSKCC Antibody and Bioresource Core Facility. All experiments were approved by the Tri-SCI Embryonic Stem Cell Research Oversight Committee (ESCRO).

### Generation of the HUES8 SOX17^{GFP/+} Reporter Line

The HUES8-iCAS9 cell line carries a puromycin resistance gene (González, 2014). Since the GECKO v2 library also relies on puromycin selection, puromycin resistance gene was knocked out by transfecting HUES8 iCas9 cells with in vitro transcribed gRNA (Table 4) that targets the puromycin resistance gene using the iCRISPR platform. A puro-sensitive cell line was identified by screening individual clones with 0.5 ug/ML puromycin treatment for 48 hours. One puro-sensitive HUES8-iCAS9 cell line was chosen (named iCas9-puroKO) for SOX17-GFP reporter targeting using the selection-free knock-in strategy previously established (Zhu, 2015) to generate the SOX17^{GFP} allele. The detailed targeting strategy was previously described here. HUES8 iCas9-puroKO cells were co-transfected with a in vitro transcribed gRNA targeting the SOX17 stop codon (FIG. 5B) and a plasmid carrying 2A-GFP flanked by homology arms. Southern blotting experiments verified one clonal cell line with the correct integration of 2A-GFP at the SOXI7 locus. The sequence for the 5′ external probe used in the southern blotting is:

(SEQ ID NO: 1)
AATCGCTAGGCCGATTTCTTAAACCCCAAACTGTTCTTTGCGAGCCTGAC
GCCCAAAACCAGGGGTGTGTAGCGGCCACGTCCTTTCTTAAGGCTCTGGG
TTCCCTTCCCGCTTCCCGCCCTCCGACCCTCCAAAGCAGCTTTCCGCCTT
GCTCTCCGGCTCCCGGATTCCCCAGGTGGCCGGGGGCGCGGGTCCAACGG
CTCTGGGAAGGCGACTTCCCGGCACCTCCGGGCGGCGCGAGAGCACCCTT
GGCCCTGAACTGGGCCGGTTGTGTCCATCCCTCGACCCCTTCCCTAGTTA
GGTGTCCTTTTCTGTTTTTCGAACGACCGGGTGATGGGTGAGCGGAAAGC
CGCTTCCAGGAGACCAAAAGAAAGGGGTGCCTTTAGAGGACGGGTGTTCC
CCAAGGGCTCGGACTCAGGAGTCCCAGATCTCCCTCTTTAACTTCACCCC
GGTTGCGCAATTCAAAGTCTGAGGGG

The probe was synthesized by PCR using the PCR DIG probe Synthesis Kit (Roche Applied Sciences, 11636090910). 20 μg genomic DNA was digested with Xmnl, which produced a 3974 bp DNA fragment with the GFP insertion, and a 3188 bp DNA fragment without the GFP insertion. Southern blotting was performed as previously described (Gonzalez, 2014).

TABLE 4
gRNA targeting sequence

| Gene | gRNA targeting sequence | SEQ ID NO |
|---|---|---|
| ACVR1B | AGCCTGAGCACCCGTCCATG | 2 |
| ACVR1B | ACACCTGCACATGGAGATCG | 3 |
| ARID1A | TTCAATAGATGACCTCCCCA | 4 |
| ARID1A | TGAGCGAGACTGAGCAACAC | 5 |
| ARID4B | TAGACATGATTCTTCAACAG | 6 |
| ARID4B | AGTTCAGGATGACCACATAA | 7 |
| ARPC4 | TACAACCTGTGACCATCAGC | 8 |
| ARPC4 | GTTTCACAGCAATGCTGACC | 9 |
| BAG6 | CATCTTGCAGAACTCGTCCC | 10 |
| BAG6 | GCAAGATGATAAGAAGCTTC | 11 |
| BANP | CAACCTCCAGATCCATCACG | 12 |
| BANP | GACCGTCCTGCCCCACGTGA | 13 |
| CCDC6 | GATTGACCTTGAAAATACAT | 14 |
| CCDC6 | CTTTCTCATAATTTACAGCA | 15 |
| CSK | TCTCCTCCACGATCACGCCC | 16 |
| CSK | CGGCTGGGCCCTGAACATGA | 17 |
| CTNNB1 | GAAACAGCTCGTTGTACCGC | 18 |
| CTNNB1 | AGAACGCATGATAGCGTGTC | 19 |
| DPH6 | CTAAGACCAGCTGAAAACCA | 20 |
| DPH6 | TCAGGAAAAAGAAGAAGTAG | 21 |
| EOMES | GGACACTCACATCGGTGTTT | 22 |
| EOMES | CCACTACAATGTGTTCGTAG | 23 |
| FOXH1 | TGGCAGAACGGAGGTGCGCG | 24 |
| FOXH1 | TGGCCGGCCGTGCAGCACGT | 25 |
| JUN | CAAGCTGGCGTCGCCCGAGC | 26 |
| JUN | GATTATCAGGCGCTCCAGCT | 27 |
| KDM1A | TATAAGGTGCTTCTAATTGT | 28 |
| KDM1A | TGTGGTCCACTGATAATATC | 29 |
| L3MBTL3 | ATGGTACCAACTGCTCAAGA | 30 |
| L3MBTL3 | TGTGAGAACTGTTGTCAGTA | 31 |
| MAPK1 | GCCTACAGACCAAATATCAA | 32 |
| MAPK1 | GCAGTAGGTCTGGTGCTCAA | 33 |
| JNK1 | CTCATAAAGTTACATAGTCA | 34 |
| JNK1 | AGAATCAGACTCATGCCAAG | 35 |
| MED12 | ACAGGTCATCTTAATGAGCC | 36 |
| MED12 | CTCAGAGATTGCTGCATAGT | 37 |
| MEKK1 | AAGGTTCTAGTTGAAACACC | 38 |
| MEKK1 | AACCTTCAGACCCAATGTTA | 39 |
| MIXL1 | AGCTCGTCTTCCGCCGGACC | 40 |
| MIXL1 | GCGCCGCGTTTCCAGCGTAC | 41 |
| MKK7 | AGGTGGTACCTGGCCCCCGA | 42 |
| MKK7 | CCTGTTCACACCCCGCAGCA | 43 |
| MLL2 | TTCCCCTGCGCGACTGCCAG | 44 |
| MLL2 | CTCACCATTGGTGTGCTGCA | 45 |
| NF2 | TGAGCCTACCTTGGCCTGGA | 46 |
| NF2 | ATTCCACGGGAAGGAGATCT | 47 |
| Non- | ACGGAGGCTAAGCGTCGCAA | 48 |
| Non- | CGCTTCCGCGGCCCGTTCAA | 49 |
| NUP188 | CGATATCCATGCCCTCCACC | 50 |
| NUP188 | CTTAAACCCAGTTCCTTCAA | 51 |
| PPP2R4 | ACCCTGGTTCACTTTGGACC | 52 |
| PPP2R4 | TTCATCCTTACCCTCAACGA | 53 |
| PTPN14 | CTAGCCGGCCTAGCTGTGCA | 54 |
| PTPN14 | GAAATAGCACATACTCTCTG | 55 |
| Puromycin | CACGCGCCACACCGTCGATC | 56 |
| SMAD2 | ATGTTATATATTGCCGATTA | 57 |
| SMAD2 | CTCCAGGTATCCCATCGAAA | 58 |
| SMAD4 | CAGGTGCCTTAGTGACCACG | 59 |
| SMAD4 | GGATTAACACTGCAGAGTAA | 60 |
| SMARCC1 | TGCTCCTACCAATAAAACAC | 61 |
| SMARCC1 | TGGGAAGCATGTCACCAACC | 62 |
| TAOK1 | TCTGCTTCGGATTTACTAGA | 63 |
| TAOK1 | ATTTACGTGAACACACAGCA | 64 |
| TGFBR1 | CCACGAACGTTCTTCTCTAG | 65 |
| TGFBR1 | CCATCGAGTGCCAAATGAAG | 66 |

### Production of the Lentivirus Library and Determination of Multiplicity of Infection (MOI)

Human CRISPR Knockout pooled library GECKO v2 (Addgene, 1000000049) was purchased from Addgene. 50 μg library plasmid with 20 μg PAX2 and 5 μg VSVG plasmid were transfected with the Jetprime (VMR, 89137972) reagent into 293T cell to pack the lentivirus. Viral supernatant was collected, filtered, aliquoted and stored at −80° C. Lentivirus infection efficiency was used to estimate the MOI according to the formula

$$P ( n ) \frac{{m}^{n} {e}^{- m}}{n 1} ,$$

where m is the MO1; n is the occurrence of event that virus enters into cells; P(n) is the probability that a cell will get infected by n viruses. The infection efficiency can be viewed as the probability of being infected which equals to 1-P(0). When MOI equals to 0.36, the infection efficiency [1-P(0)] is 30%, and the probability of a cell getting 2 or more viruses is 16.28%. To determine the viral titer for an infection efficiency of 30%, 0.15 million SOX17^{GFP/+} ES cells per well were infected with different amounts of virus (0-20 pl) in duplicates in six-well plates. 48 hours later, puromycin (0.5 μg/ml) was added into one set of the cells to select infected cells. After 48 hours treatment of puromycin, control uninfected cells were killed by puromycin selection. The ratio of the cell number of the selected set (treated with puromycin) over the unselected set (not treated with puromycin) was calculated to determine the infection efficiency. The amount of virus needed for the 30% infection efficiency in the six-well format is scalable to 150 mm plate by a factor of 15.

### Genome-Wide Lentivrial CRISPR Screen

A minimum 200-fold library coverage is typically recommended for screens based on basic phenotypes such as cell survival and growth. Based on the more complex nature of this screen focused on lineage decisions, A ˜1,000-fold coverage was targeted to maximize sensitivity. ˜200 million iCas9 SOX17^{GFP/+} HUES8 cells were collected after splitting, then infected by the lentiviral library with a low MOI of 0.36 at Day 0 in 150 mm plates (100 plates total). 6 pg/ml protamine sulfate was added on the first day of infection to enhance the infection efficiency. Doxcycline (2 ug/ml) was added from day1-day7. Puromycin (0.5 ug/ml) was added from day2-day7. At day 7, cell were treated with TrpLE Select (Thermo Fisher Scientific, 12563029) and 120 million cells were plated into 150 mm plates (59 plates total) for DE differentiation. On Day 8, cells were switched from the maintenance E8 medium to the DE differentiation medium (described in the “Definitive endoderm differentiation” subsection). After 3 days of DE differentiation (Day 8 to Day 11), DE cells were splited by TrpLE Select and sorted by FACS Aria according to GFP positive and negative expression. Sorted cells were pelleted and genomic DNA were extracted using QiAGEN blood & cell culture DNA maxi kit (Cat No. 13362) immediately after sorting. Genomic DNA were sent to MSKCC RNAi core for Hi-SEQ library preparation.

### Hi-Seq and Data Analysis

A two-step PCR method was performed to amplify the gRNA sequence for Hi-Seq. For the first step, 380 pg of DNA per sample (6.6 pg of genomic DNA per 1 million cells) was used to perform PCR using Herculase II fusion DNA polymerase (Agilent, 600679) in order to achieve a 1,000-fold converge of the GECKO library containing 58,028 gRNAs. Primers sequences to amplify lentiCRISPR gRNAs for the first PCR are:

(SEQ ID NO: 67)
F1: AATGGACTATCATATGCTTACCGTAACTTGAAAGTATTTCG
(SEQ ID NO: 68)
R1: CTTTAGTTTGTATGTCTGTTGCTATTATGTCTACTATTCTTTCC.

In total, 38 separate 100 μl reaction with 10 μg genomic DNA per sample for 18 cycles and combined the resulting amplicons were performed. For the second step, 5 pl of the product from the first PCR was used in a 100 pl PCR reaction for 24 cycles with primers to attach Illumina adaptors for barcoding. Primers used in this reaction are:

(SEQ ID NO: 69)
F2: AATGATACGGCGACCACCGAGATCTACACTCTTTCCCTACACGACG
CTCTTCCGATCT(1-9 bp variable length sequence)tcttg
tggaaaggacgaaacaccg
(SEQ ID NO: 70)
R2: CAAGCAGAAGACGGCATACGAGAT (6 bp barcode)
GTGACTGGAGTTCAGACGTGTGCTCTTCCGATCTtctactattattcccc
tgcactgt

Gel purified amplicons from the second PCR were quantified, mixed and sequenced using a HiSeq 2500 (Illumina) at MKSCC Integrated Genomics Operation (IGO). Raw FASTQ files demultiplexed by MSKCC IGO were further processed to contain only the unique gRNA sequences, and the processed reads were aligned to the designed gRNA sequences from the library using the FASTX-Toolkit (http://hannonlab.cshl.edu/fastx toolkit/). The read counts were further normalized to total reads of that sample to offset differences in Hi-Seq depth. Z-score of each gRNA and gene was calculated as illustrated in FIG. 6A. Gene ontology of positive and negative regulator gene hits was analyzed through Panther Classification System.

### Hit Validation and Generation of Clonal KO Human ES Cell Lines

gRNAs were cloned into IentiCRISPR v2 (Addgene, 52961) following protocols provided by Addgene. 1 μg IentiCRISPR, 0.1 μg VSVG and 0.4 μg PAX2 plasmids were transfected with the Jetprime (VMR, 89137972) reagent into 293T cells to pack lentiviruses. Viral supernatant was collected, filtered, aliquoted and stored at −80° C. A MOI of 0.36 or less was used for the infection of the H1 iCas9 cell line with different IentiCRISPR viruses. Cells were treated with 2 pg/ml doxycycline (Day 1 to 7) and 0.5 μg/ml puromycin (Day 2 to 7). On Day 7, cells were treated with TrypLE Select and 0.1 million cells per well were plated in duplicate sets in a six-well plate. One set was used for definitive endoderm differentiation the next day. The other set was cultured in E8 media for maintenance until the second repeat of differentiation. gRNA targeting sequences selected from GECKO v2 library are listed in Table 4. Two gRNAs per gene were tested for validation. Two non-targeting gRNAs were used as WT control.

### Generation of Clonal KO Human ES Cell Lines

Clonal KO lines were created as previously described (González, 2014) with some modifications. H1 iCas9 cells were infected with lentiviruses expressing MKK7 or JUN targeting gRNAs made from the lentiCRISPR v2 construct (Table 4) on Day 0. Next, cells were treated with 2 μg/ml doxycycline (Day 1 to 7) and 0.5 μg/ml puromycin (Day 2 to 7). On Day 7, infected ES cells were dissociated to single cells using TrypLE Select. 500 cells were plated into one 100 mm tissue culture dish with 10 ml E8 media supplemented with 10 μm ROCK inhibitor (Selleck Chemicals, 1254) for colony formation. After expanding the clonal cell colonies for 10 days, 50 ES cell colonies were picked from the 100 mm tissue culture dish into a 96-well plate. Genomic DNA was extracted for PCR genotyping. Primers used for PCR and sequencing are listed in Table 3.

TABLE 3
Genotyping primer information

| Gene | Forward Primer (5′ to 3′) | Reverse primer (5′ to 3′) | Sequencing Primer (5′ to 3′) |
|---|---|---|---|
| MKK7 | AGCCTCCTCCATCTCTTTCC | ATGAGGATGCGCTTGTTCTC | GATGAGACAAGGGACCCTGA |
| | (SEQ ID NO: 71) | (SEQ ID NO: 73) | (SEQ ID NO: 75) |
| JUN | ACTTTTCAAAGCCGGGTAGC | CACTGTCTGAGGCTCCTCCT | TGACTGCAAAGATGGAAACG |
| | (SEQ ID NO: 72) | (SEQ ID NO: 74) | (SEQ ID NO: 76) |

### Definitive Endoderm Differentiation

Human ES or iPS cells were cultured in E8 medium and routinely passed by EDTA. When cells reach to 80-90% confluences, cells were treated with TrpLE Select and get single cell for passaging. Typically, 0.15 millions human ES or iPS cells were plated in one well of the six-well plates with ROCK inhibitor 10 uM in 2 ML E8 media. For the H1 line, it was typically plated 0.1 million cells to accommodate the higher proliferation rate. Twenty-four hours later, cells were washed with PBS once and add Day0 media (Advanced RPMI (Thermo Fisher Scientific, 12633012) with penicillin/streptomycin (Thermo Fisher Scientific, 15070063), GlutaMAX (Thermo Fisher Scientific, 35050079), 0.003% BSA (Thermo Fisher Scientific, 15260037), 5 pM CHIR-99021 (Tocris, 4423) and 100 ng/ml Activin A (PeperoTech, 12014E)). Over the next 2 days, cells were changed to Day2 media (Advanced RPMI with penicillin/streptomycin, GlutaMAX, 0.2% FBS and 100 ng/ml Activin A). 1 pm JNK inhibitor JNK-IN-8 (Selleckchem, S4901) was used during differentiation when indicated. Routinely, around 60-80% SOX17+ cells were obtained in 3 days.

### Neuroectoderm Differentiation

Human ES cells cultured in E8 were disaggregated using TrypLE Select for 5 minutes and washed using E8 media. The cells were plated on Matrigel (BD, 354234) coated dishes in E8 media with ROCK-inhibitor at a density of 180,000-200,000 cells/cm². After 1 day of culture in E8 media differentiation to neuroectoderm was initiated by switching to knockout serum replacement (KSR) media with 10 μM TGF-β receptor inhibitor SB431542 (Tocris, 161410) and 100 nM BMP inhibitor LDN193189 (Axon Medchem, 1509). On Day 1 and Day 2 of differentiation, the media was removed and fresh KSR with 10 μM SB431542 and 100 nM LDN193189 was added. Starting on day 4 of differentiation an increasing amount of N2 media was added to the KSR media every two days, while maintaining 10 μM SB431542 and 100 nM LDN193189. On Day 4 a 3:1 mixture of KSR/N2 media was added. On Day 6 a 1:1 mixture of KSR/N2 media was added and on day 8, a 1:3 mixture of KSR/N2 media was added. The cells were isolated for flow cytometry analysis on Days 4, 6, 8 and 10 of differentiation and for immunostaining on Day 10 of differentiation. KSR media contains Knockout DMEM (Thermo Fisher Scientific, 10829018), Knockout Serum Replacement (Thermo Fisher Scientific, 10828028), 1×MEM Non-Essential Amino Acids (Thermo Fisher Scientific, 11140050), 1× GlutaMAX (Thermo Fisher Scientific, 35050079), and 2-mercaptoethanol (Thermo Fisher Scientific, 21985023). N2 media contains DMEM/F12 medium (Thermo Fisher Scientific, 12500¬062), glucose (Sigma, G8270), sodium bicarbonate (Sigma, S5761), putrescine (Sigma, P5780), progesterone (Sigma, P8783), sodium selenite (Sigma, S5261), apo-transferrin (Sigma, T1147), and insulin (Sigma, 12643). Western Blot

Cell pellets were quickly snap frozen in liquid nitrogen and lysated in lysis buffer (Cell Signaling Technology, 9803) with proteinase/phosphatase inhibitors (Cell Signaling Technology, 5872) and 1 mm PMSF (MP Biomedicals, ICN19538105). Proteins were pre-cleared by centrifugation at 14,000 g 4° C. for 10 minutes. Protein concentration was determined by Bradford assay (Bio-Rad, 500-0202). Equal amounts of protein were loaded into Bis-Tris 10% gel (Novex, NP0301BOX) and transferred to nitrocellulose membranes (Novex, LC2001). Membranes were blocked with 5% milk (for non-phosphorylated proteins) or 5% BSA (for phosphorylated proteins). Primary antibody was incubated overnight at 4° C. Membranes were washed with TBST 3 times for 10 minutes each and incubated with secondary antibody for 1 hour at R.T. Membrane were washed with TBST 3 times for 10 minutes each. ECL western blotting detection reagents (Amersham, RPN2236 and Thermo Fisher Scientific, 32106) were used to visualize the protein bands. All antibodies and dilution factors are listed in Table 1.

TABLE 1
Antibody information

| Protein | Application | Species | Vendor | Cat. Number | Dilution |
|---|---|---|---|---|---|
| MKK7 | WB | rabbit | Cell Signaling Techology | 4172S | 1:1,000 |
| C-JUN | WB | rabbit | Cell Signaling Techology | 9165S | 1:1,000 |
| P-JUN S63 | WB | rabbit | Cell Signaling Techology | 9261S | 1:500 |
| P-JUN S73 | WB | rabbit | Cell Signaling Techology | 3270S | 1:1,000 |
| P-SMAD2 | WB | rabbit | Cell Signaling Techology | 3108S | 1:500 |
| SMAD2 | WB | rabbit | Cell Signaling Techology | 5339S | 1:1,000 |
| JNK1 | WB | mouse | Cell Signaling Techology | 3706S | 1:1,000 |
| P-JNK | WB | mouse | Cell Signaling Techology | 9255S | 1:500 |
| GAPDH | WB | rabbit | Cell Signaling Techology | 5174S | 1:25,000 |
| anti-rabbit HRP | WB | | Cell Signaling Techology | 7074s | 1:5,000 |
| anti-mouse HRP | WB | | Cell Signaling Techology | 7076s | 1:5,000 |
| SOX17 | Flow | mouse | BD BIOSCIENCES | 561591 | 1:50 |
| CXCR4 | Flow | mouse | R&D Systems | FAB170A | 1:25 |
| PAX6 | Flow/IF | rabbit | Covance | PRB278P100 | 1:50 |
| GATA4 | Flow | mouse | BD Biosciences | 560400 | 1:50 |
| GATA6 | Flow | rabbit | Cell Signaling Techology | 26452s | 1:50 |
| Phospho Histone H3 | Flow | rabbit | Cell Signaling Techology | 3465S | 1:50 |
| Cleaved Caspase 3 | Flow | rabbit | Cell Signaling Techology | 9602s | 1:50 |
| SOX17 | IF | goat | R&D Systems | AF1924 | 1:500 |
| FOXA2 | IF | rabbit | Millipore | 7633 | 1:500 |
| OCT4 | IF | goat | Santa Cruz | sc8628 | 1:500 |
| NANOG | IF | rabbit | Cosmobio Japan | RECRCAB0004PF | 1:500 |
| GFP | Flow/IF | rat | Biolegend | 338008 | 1:100 |
| Alexa Fluor 488 | IF | goat | Thermo Fisher Scientific | A11055 | 1:500 |
| Alexa Fluor 488 | IF | rat | Thermo Fisher Scientific | A21208 | 1:500 |
| Alexa Fluor 594 | IF | rabbit | Thermo Fisher Scientific | A21207 | 1:500 |
| Alexa Fluor 594 | IF | goat | Thermo Fisher Scientific | A11058 | 1:500 |
| Alexa Fluor 647 | IF | rabbit | Thermo Fisher Scientific | A31573 | 1:500 |

WB: western blotting; IF: immunofluorescence staining; Flow: flow cytometry.

### Flow Cytometry

Cells were dissociated using TrypLE Select and resuspended in FACS buffer (5% FBS, 5 mM EDTA in PBS). First, cells were stained with surface antibody (CXCR4-APC) with LIVE/DEAD violet dye (Molecular Probe, L34955, 1:1,000) for 30 minutes at 4° C. After washing, cells were fixed and permeabilized in 1× fix/perm buffer for 30 mins 4 C. After washing with FACS buffer, cells were fixed and permeabilized in 1× fixation/permeabilization buffer (eBioscience, 00-5523-00) for 30 minutes at 4° C. After fixation and permeabilization, cells were stained with intracellular conjugated antibody (SOX17-PE, GATA6-PE, GATA4-Alexa-647) in permeabilization buffer (eBioscience, 00-5523-00) for 30 minutes at 4° C. After washing with permeabilization buffer, cells were resuspended in FACS buffer, and samples were analyzed using BD LSRfortessa or BD LSRII. Flow cytometry analysis and figures were generated in Flowjo 10. Flow cytometry graded antibodies are listed in Table 1.

### RNA Isolation and RT-qPCR

Cell pellets were lysed in TRIzol (Thermo Fisher Scientific, 15596018). RNA was extracted from TRIzol lysate using the RNeasy Mini Kit (Qiagen, #74106). Then cDNA was produced using High Capacity cDNA Reverse Transcription Kit (Applied Biosystems, #4368814). Quantitative real-time PCR was performed in triplicate using ABsolute Blue QPCR SYBR Green Mix with low ROX (Thermo Scientific, #AB4322B) on the ABI PRISM® 7500 Real Time PCR System (Applied Biosystems) using the following protocol: 15 minutes at 95° C. followed by 40 cycles of 15 seconds at 95° C., 30 seconds at 58° C., and 30 seconds at 72° C. The signal was detected at 72° C. All primers used for RT-qPCR were listed in Table 2.

TABLE 2
qPCR primers

| Gene | Forward Primer (5′ to 3′) | Reverse primer (5′ to 3′) |
|---|---|---|
| OCT4 | AGTGAGAGGCAACCTGGAGA (SEQ ID NO: 77) | ACACTCGGACCACATCCTTC (SEQ ID NO: 86) |
| NANOG | CATGAGTGTGGATCCAGCTTG (SEQ ID NO: 78) | CCTGAATAAGCAGATCCATGG (SEQ ID NO: 87) |
| SOX2 | TGGACAGTTACGCGCACAT (SEQ ID NO: 79) | CGAGTAGGACATGCTGTAGGT (SEQ ID NO: 88) |
| SOX17 | CGCACGGAATTTGAACAGTA (SEQ ID NO: 80) | GGATCAGGGACCTGTCACAC (SEQ ID NO: 89) |
| EOMES | CAACATAAACGGACTCAATCCCA (SEQ ID NO: 81) | ACCACCTCTACGAACACATTGT (SEQ ID NO: 90) |
| GATA6 | CCCACAACACAACCTACAGC (SEQ ID NO: 82) | GCGAGACTGACGCCTATGTA (SEQ ID NO: 91) |
| GATA4 | TCCCTCTTCCCTCCTCAAAT (SEQ ID NO: 83) | TCAGCGTGTAAAGGCATCTG (SEQ ID NO: 92) |
| FOXA2 | GGGAGCGGTGAAGATGGA (SEQ ID NO: 84) | TCATGTTGCTCACGGAGGAGTA (SEQ ID NO: 93) |
| GAPDH | GGAGCCAAACGGGTCATCATCTC (SEQ ID NO: 85) | GAGGGGCCATCCACAGTCTTCT (SEQ ID NO: 94) |
| ChIP-region | Forward Primer (5′ to 3′) | Reverse primer (5′ to 3′) |
| SOX17 enhancer | CTCCTGGCTCCAGGTGATAG | TCCCCTGTGTTTGGAGAAAG |
| upstream | (SEQ ID NO: 95) | (SEQ ID NO: 98) |
| GATA6 enhancer | TCCAACAGTCCCCTGATTTC | CCTCAAGCTGCTCCCAGATA |
| upstream | (SEQ ID NO: 96) | (SEQ ID NO: 99) |
| SOX17 negative | TGGCCAGGCATGGTGTAATC | ATGTTGGCCAGGCTGGTCTC |
| control region | (SEQ ID NO: 97) | (SEQ ID NO: 100) |
| (Teo et.al) | | |

### Immunostaining

Cells were fixed by 4% paraformaldehyde (Thermo Fisher Scientific, 50980495) for 10 mins at room temperature. After washing with PBST (PBS with 0.2% Triton) three times for 5 minutes each, cells were blocked in 5% donkey serum in PBST buffer for 5 minutes. Cells were incubated with primary antibodies for 1 hour at RT. After washing with PBST three times for 5 minutes each, cells were incubated with fluoresce conjugated secondary antibodies and 0.2 pg/ml DAPI (Sigma, 32670-5 mg-F) for 1 hour at RT. After washing with PBST three times for 5 minutes each, images were taken with equal exposure for the same field. All primary and secondary antibodies used are listed in Table 1.

### Chromatin Immunoprecipitation

Definitive endoderm cells at Day 2 were collected for chromatin immunoprecipitation. Cells were cross-linked with 1% formaldehyde (Sigma, F1635) at 37° C. for 15 min and quenched with 0.125 M glycine for 5 min at room temperature. ChIP was performed as previously described 52. Samples were incubated with 3-5 pg of antibody bound to 60 pl Dynabeads protein G (Thermo Fisher Scientific, 1004D), then incubated overnight at 4° C. 2% pre-cleared chromatin prior to primary antibody addition was kept as input DNA. Magnetic beads were washed and chromatin was eluted, and reverse cross-linked ChIP DNA was dissolved in 10 mM Tris pH 8.0 buffer for further analysis. For ChIP-qPCR, immunoprecipitated DNA was analyzed by qPCR, and the amplification product was expressed as percentage of the 2% input. PCR primer pairs used to amplify the negative control and enhancer regions of indicated genes are listed in Table 2.

### Statistical Methods

Student T-test, unpaired, two-tail analysis was used to calculate the p value.

### 6.2. Example 2: Transient Inhibition of JNK Pathway Improved Differentiation Efficiency of Endoderm Derivatives

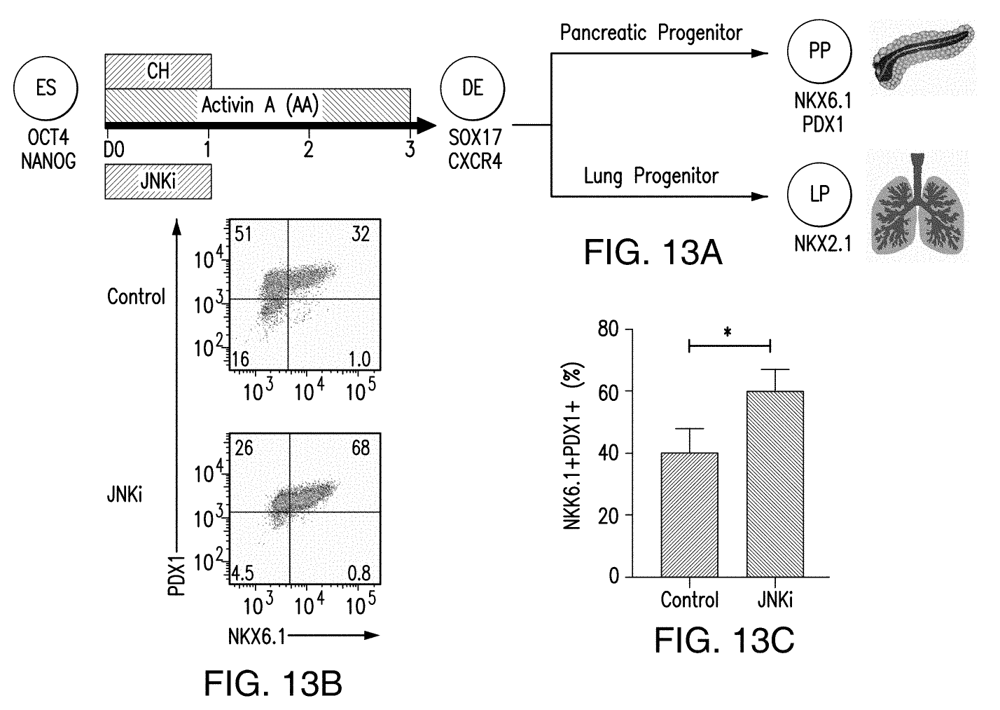

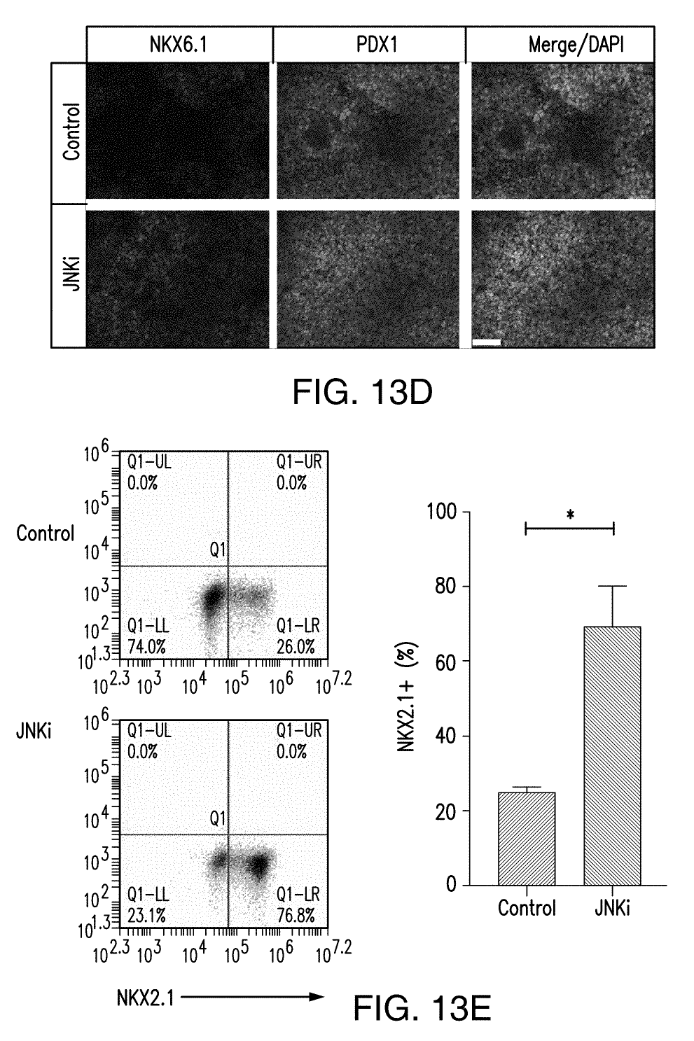

An improved protocol suitable for endoderm derivative lineage differentiation was developed. The improved protocol dispensed with the use of serum and inhibited JNK on the first day of definitive endoderm differentiation (FIG. 13A). It was observed that one day of JNK inhibition was sufficient to induce differentiation of definitive endoderm, as evidenced by SOX17 and CXCR4 expression (FIG. 13A). This transient inhibition of the JNK pathway also improved pancreatic progenitor and lung progenitor differentiation from the differentiated definitive endoderm. Flow cytometry and immunostaining analysis showed that the number of NKX6.1 and PDX1 expressing pancreatic progenitor cells, and NKX2.1 expressing lung progenitor cells, differentiated from endoderm were significantly increased when the endoderm was differentiated by transiently inhibiting the JNK pathway (FIGS. 13B-13E). These results showed that the JNKi-induced definitive endoderm cells were functionally similar to untreated endoderm cells, but had superior potential for efficiently differentiating into endoderm lineage derivatives.

### REFERENCES

Anderson, K. V. & Ingham, P. W. The transformation of the model organism: a decade of developmental genetics. Nat Genet 33 Suppl, 285-293 (2003).
Angel, P., Hattori, K., Smeal, T. & Karin, M. The jun proto-oncogene is positively autoregulated by its product, Jun/AP-1. Cell 55, 875-885 (1998).
Arai, S. & Miyazaki, T. Impaired maturation of myeloid progenitors in mice lacking novel Polycomb group protein MBT-1. EMBO J 24, 1863-1873 (2005).
Arnold, S. J., Hofmann, U. K., Bikoff, E. K. & Robertson, E. J. Pivotal roles for eomesodermin during axis formation, epithelium-to-mesenchyme transition and endoderm specification in the mouse. Development 135, 501-511 (2008).
Arnulf, B. et al. Human T-cell lymphotropic virus oncoprotein Tax represses TGF-beta 1 signaling in human T cells via c-Jun activation: a potential mechanism of HTLV-I leukemogenesis. Blood 100, 4129-4138 (2002).
Beyer, T. A. et al. Switch Enhancers Interpret TGF-band Hippo Signaling to Control Cell Fate in Human Embryonic Stem Cells. CellReports 1-14 (2013). doi:10.1016/j.celrep.2013.11.021
Bock, C. et al. Reference Maps of Human ES and iPS Cell Variation Enable High-Throughput Characterization of Pluripotent Cell Lines. Cell 144, 439-452 (2011).
Brown, S. et al. Activin/Nodal Signaling Controls Divergent Transcriptional Networks in Human Embryonic Stem Cells and in Endoderm Progenitors. Stem Cells 29, 1176-1185 (2011).
Canver, M. C. et al. BCL11A enhancer dissection by Cas9-mediated in situ saturating mutagenesis. Nature 527, 192-197 (2015).
Chambers, S. M. et al. Highly efficient neural conversion of human ES and iPS cells by dual inhibition of SMAD signaling. Nat Biotechnol 27, 275-280 (2009).
Chia, N.-Y. et al. A genome-wide RNAi screen reveals determinants of human embryonic stem cell identity. Nature 468, 316-320 (2010).
Costello, I. et al. The T-box transcription factor Eomesodermin acts upstream of Mesp1 to specify cardiac mesoderm during mouse gastrulation. Nat Cell Biol 13, 1084-1091 (2011).
D'Amour, K. A. et al. Efficient differentiation of human embryonic stem cells to definitive endoderm. Nat Biotechnol 23, 1534-1541 (2005).
Davis, R. J. Signal Transduction by the JNK Group of MAP Kinases. Cell 103, 239-252 (2000).
Deglincerti, A. et al. Self-organization of the in vitro attached human embryo. Nature 533, 251-254 (2016).
Estaras, C., Benner, C. & Jones, K. A. SMADs and YAP Compete to Control Elongation of 13-Catenin:LEF-1-Recruited RNAPII during hESC Differentiation. Molecular Cell 58, 780-793 (2015).
Gonzales, K. A. U. et al. Deterministic Restriction on Pluripotent State Dissolution by Cell-Cycle Pathways. Cell 162, 564-579 (2015).
Gonzalez, F. CRISPR/Cas9 genome editing in human pluripotent stem cells: Harnessing human genetics in a dish. Dev. Dyn. 245, 788-806 (2016).
González, F. et al. An iCRISPR platform for rapid, multiplexable, and inducible genome editing in human pluripotent stem cells. Cell Stem Cell 15, 215-226 (2014).
Gu, Z. et al. The type I serine/threonine kinase receptor ActRIA (ALK2) is required for gastrulation of the mouse embryo. Development 126, 2551-2561 (1999).
Haegel, H. et al. Lack of beta-catenin affects mouse development at gastrulation. Development 121, 3529-3537 (1995).
Hart, A. H. et al. Mixll is required for axial mesendoderm morphogenesis and patterning in the murine embryo. Development 129, 3597-3608 (2002).
Iratni, R. et al. Inhibition of excess nodal signaling during mouse gastrulation by the transcriptional corepressor DRAP1. Science 298, 1996-1999 (2002).
Javelaud, D. & Mauviel, A. Crosstalk mechanisms between the mitogen-activated protein kinase pathways and Smad signaling downstream of TGF-[beta]: implications for carcinogenesis. Oncogene 24, 5742-5750 (2005).
Johnson, R. & Halder, G. The two faces of Hippo: targeting the Hippo pathway for regenerative medicine and cancer treatment. Nat Rev Drug Discov 13, 63-79 (2014)
Kagey, M. H. et al. Mediator and Cohesin Connect Gene Expression and Chromatin Architecture. Nature 467, 430-435 (2010).
Kanai-Azuma, M. et al. Depletion of definitive gut endoderm in Sox17-null mutant mice. Development 129, 2367-2379 (2002).
Kim, S. W. et al. Chromatin and transcriptional signatures for Nodal signaling during endoderm formation in hESCs. Developmental Biology 357, 492-504 (2011).
Korkmaz, G. et al. Functional genetic screens for enhancer elements in the human genome using CRISPR-Cas9. Nat Biotechnol 34, 192-198 (2016).
Kubo, A. Development of definitive endoderm from embryonic stem cells in culture. Development 131, 1651-1662 (2004).
Larsson, J. et al. Abnormal angiogenesis but intact hematopoietic potential in TGF-beta type I receptor-deficient mice. EMBO J 20, 1663-1673 (2001).
Liu, S. J. et al. CRISPRi-based genome-scale identification of functional long noncoding RNA loci in human cells. Science 355, (2017).
Massague, J. TGFI3 signalling in context. Nature Reviews Molecular Cell Biology 13, 616-630 (2012).
Meno, C. et al. Mouse Lefty2 and zebrafish antivin are feedback inhibitors of nodal signaling during vertebrate gastrulation. Molecular Cell 4, 287-298 (1999).
Nomura, M. & Li, E. Smad2 role in mesoderm formation, left-right patterning and craniofacial development. Nature 393, 786-790 (1998).
Nüsslein-Volhard, C. & Wieschaus, E. Mutations affecting segment number and polarity in Drosophila. Nature 287, 795-801 (1980).
Osafune, K. et al. Marked differences in differentiation propensity among human embryonic stem cell lines. Nat Biotechnol 26, 313-315 (2008).
Parnas, O. et al. A Genome-wide CRISPR Screen in Primary Immune Cells to Dissect Regulatory Networks. Cell 162, 675-686 (2015).
Pauklin, S. & Vallier, L. Activin/Nodal signalling in stem cells. Development 142, 607-619 (2015).
Perea-Gomez, A. et al. Nodal antagonists in the anterior visceral endoderm prevent the formation of multiple primitive streaks. Developmental Cell 3, 745-756 (2002).
Pessah, M. et al. c-Jun associates with the oncoprotein Ski and suppresses Smad2 transcriptional activity. J Blot Chem 277, 29094-29100 (2002).
Pessah, M. et al. c-Jun interacts with the corepressor TG-interacting factor (TGIF) to suppress Smad2 transcriptional activity. Proc Natl Acad Sci USA 98, 6198¬6203 (2001).
Plouffe, S. W. et al. Characterization of Hippo Pathway Components by Gene Inactivation. Molecular Cell 64, 993-1008 (2016).
Ribeiro, P. S. et al. Combined Functional Genomic and Proteomic Approaches Identify a PP2A Complex as a Negative Regulator of Hippo Signaling. Molecular Cell 39, 521-534 (2010).
Robertson, E. J. Dose-dependent Nodal/Smad signals pattern the early mouse embryo. Seminars in Cell and Developmental Biology 32, 73-79 (2014).
Sanjana, N. E., Shalem, O. & Zhang, F. Improved vectors and genome-wide libraries for CRISPR screening. Nature Methods 11, 783-784 (2014).
Schier, A. F. Nodal Signaling in Vertebrate Development. Annu. Rev. Cell Dev. Biol. 19, 589-621 (2003).
Shalem, O. et al. Genome-Scale CRISPR-Cas9 Knockout Screening in Human Cells. Science 343, 84-87 (2014).
Shi, Z.-D. et a/. Genome Editing in hPSCs Reveals GATA6 Haploinsufficiency and a Genetic Interaction with GATA4 in Human Pancreatic Development. Cell Stem Cell (2017). doi:10.1016/j.stem.2017.01.001
Sirard, C. et al. The tumor suppressor gene Smad4/Dpc4 is required for gastrulation and later for anterior development of the mouse embryo. Genes & Development 12, 107-119 (1998).
Tada, S. et al. Characterization of mesendoderm: a diverging point of the definitive endoderm and mesoderm in embryonic stem cell differentiation culture. Development 132, 4363-4374 (2005).
Tam, P. P. L. & Loebel, D. A. F. Gene function in mouse embryogenesis: get set for gastrulation. Nat Rev Genet 8, 368-381 (2007).
Tan, J. Y., Sriram, G., Rufaihah, A. J., Neoh, K. G. & Cao, T. Efficient Derivation of Lateral Plate and Paraxial Mesoderm Subtypes from Human Embryonic Stem Cells Through GSKi-Mediated Differentiation. Stem Cells and Development 22, 1893-1906 (2013).
Teo, A. K. K. et al. Pluripotency factors regulate definitive endoderm specification through eomesodermin. Genes & Development 25, 238-250 (2011).
Tsankov, A. M. et al. Transcription factor binding dynamics during human ES cell differentiation. Nature 518, 344-349 (2015).
Uthman, S. et al. The Amidation Step of Diphthamide Biosynthesis in Yeast Requires DPH6, a Gene Identified through Mining the DPH1-DPH5 Interaction Network. PLoS Genet 9, e1003334 (2013).
van Amerongen, R. & Berns, A. Knockout mouse models to study Wnt signal transduction. Trends Genet 22, 678-689 (2006).
Wang, H., Shen, Q., Ye, L.-H. & Ye, J. MED12 mutations in human diseases. Protein & Cell 4, 643-646 (2013).
Wang, P., Rodriguez, R. T., Wang, J., Ghodasara, A. & Kim, S. K. Targeting SOX17 in human embryonic stem cells creates unique strategies for isolating and analyzing developing endoderm. Cell Stem Cell 8, 335-346 (2011).
Wang, Q. et al. The p53 Family Coordinates Wnt and Nodal Inputs in Mesendodermal Differentiation of Embryonic Stem Cells. Cell Stem Cell 20, 70-86 (2017).
Xu, R.-H. et al. NANOG is a direct target of TGFbeta/activin-mediated SMAD signaling in human ESCs. Cell Stem Cell 3, 196-206 (2008).
Yamamoto, M. et al. The transcription factor FoxH1 (FAST) mediates Nodal signaling during anterior-posterior patterning and node formation in the mouse. Genes & Development 15, 1242-1256 (2001).
Zhang, T. et al. Discovery of potent and selective covalent inhibitors of JNK. Chem Biol 19, 140-154 (2012).
Zhou, X., Sasaki, H., Lowe, L., Hogan, B. L. & Kuehn, M. R. Nodal is a novel TGF-beta-like gene expressed in the mouse node during gastrulation. Nature 361, 543-547 (1993).
Zhu, Z. & Huangfu, D. Human pluripotent stem cells: an emerging model in developmental biology. Development 140, 705-717 (2013).
Zhu, Z. et al. Genome Editing of Lineage Determinants in Human Pluripotent Stem Cells Reveals Mechanisms of Pancreatic Development and Diabetes. Cell Stem Cell 18, 755-768 (2016).
Zhu, Z., Verma, N., Gonzalez, F., Shi, Z.-D. & Huangfu, D. A CRISPR/Cas-Mediated Selection-free Knockin Strategy in Human Embryonic Stem Cells. Stem Cell Report's 4, 1103-1111 (2015).
Zorn, A. M. & Wells, J. M. Vertebrate Endoderm Development and Organ Formation. Annu. Rev. Cell Dev. Biol. 25, 221-251 (2009).

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

What is claimed is:

1. An in vitro method for differentiating pluripotent cells comprising:
contacting a plurality of pluripotent cells with at least one inhibitor of JUN N-terminal Kinase (JNK) pathway signaling, wherein the at least one inhibitor of JNK signaling is contacted to the plurality of pluripotent cells in an amount and for a period of time, such that the plurality of cells differentiate and express one or more of SRY-box 17 (SOX17), forkhead box protein A2 (FOXA2), C-X-C motif chemokine receptor 4 (CXCR4), eomesodermin (EMOES), GATA binding protein 4 (GATA4), and GATA binding protein 6 (GATA6).

2. The method of claim 1, wherein the cells are contacted with at least one activator of Wingless (Wnt) signaling and one activator of Nodal signaling.

3. The method of claim 2, wherein the at least one activator of Wnt signaling is contacted to the plurality of pluripotent cells for at least about 1 day, and wherein the at least one activator of Nodal signaling is contacted to the plurality of pluripotent cells for at least about 3 days.

4. The method of claim 1, wherein the at least one inhibitor of JNK signaling is contacted to the plurality of pluripotent cells for at least about 3 days.

5. The method of claim 1, wherein the at least one inhibitor of JNK signaling is contacted to the plurality of pluripotent cells at a concentration of at least about 0.5 μM.

6. The method of claim 2, wherein the at least one activator of Wnt signaling is contacted to the plurality of pluripotent cells at a concentration of at least about 4.5 μM.

7. The method of claim 2, wherein the at least one activator of Nodal signaling is contacted to the plurality of pluripotent cells at a concentration of at least about 4.5 ng/mL.

8. The method of claim 1, wherein the JNK signaling inhibitor is an inhibitor of a gene selected from the group consisting of mitogen-activated protein kinase 1 (MEKK1), mitogen-activated protein kinase 7 (MKK7), mitogen-activated protein kinase 4 (MKK4), c-Jun N-terminal kinase 1 (JNK1), Jun proto-oncogene (C-JUN), and combinations thereof.

9. The method of claim 1, wherein the JNK signaling inhibitor is selected from the group consisting of JNK-IN-8, SP600125, JNK Inhibitor IX, DTP3, and combinations thereof.

10. The method of claim 1, wherein the method further comprises subjecting the population of differentiated cells to conditions favoring maturation of the cells into insulin-secreting β cells.

11. The method of claim 1, wherein the pluripotent cells are selected from the group consisting of human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

12. The method of claim 1, wherein the at least one activator of Wnt signaling comprises CHIR99021, Wnt3A, and/or Wnt1.

13. The method of claim 1, wherein the at least one activator of Nodal signaling comprises Activin A.

14. An endodermal-derived cell, or precursor thereof, differentiated according to the method of claim 1.

15. The endodermal-derived cell, or precursor thereof, of claim 14, wherein the endodermal-derived cell, or precursor thereof, is a recombinant cell that expresses a detectable marker.

16. A kit comprising the endodermal-derived cell, or precursor thereof, of claim 14.

17. A composition comprising the endodermal-derived cell, or precursor thereof, of claim 14.

18. The composition of claim 17, further comprising a biocompatible scaffold.

19. A kit for differentiating pluripotent cells into endodermal cells comprising a JNK signaling inhibitor and instructions for contacting the JNK signaling inhibitor to a plurality of pluripotent cells in an amount and for a period of time, such that the plurality of cells differentiate and express one or more of SOX17, FOXA2, CXCR4, EMOES, GATA4, and GATA6.

20. A method of treating a disorder of endoderm-derived cells, tissues or organs in a subject in need thereof, comprising administering the endodermal-derived cell, or precursor thereof, of claim 14 to the subject.

21. The method of claim 20, wherein the disorder of endoderm-derived cells, tissues or organs is diabetes, and wherein the endodermal-derived cell, or precursor thereof, is administered in an amount effective to decrease one or more symptoms of diabetes.
